(19) United States
(12) Patent Application Publication
(10) Pub. No.: US 2025/0019454 A1
(43) Pub. Date: Jan. 16, 2025

(54) ANTI-TNFR2 ANTIBODIES AND USES THEREOF

(71) Applicant: Biocytogen Pharmaceuticals (Beijing) Co., Ltd., Beijing (CN)

(72) Inventors: Yongfei Yang, Beijing (CN); Shuzhen Cao, Beijing (CN); Jing Zhang, Beijing (CN); Zhe Shao, Beijing (CN); Xueyuan Jiang, Beijing (CN)

(21) Appl. No.: 18/712,043

(22) PCT Filed: Dec. 22, 2022
(86) PCT No.: PCT/CN2022/140958

§ 371 Date: May 21, 2024

(30) Foreign Application Priority Data
Dec. 22, 2021 (WO) PCT/CN2021/140487

(51) Int. Cl. C07K 16/28; A61K 39/00; A61P 35/00

(52) U.S. Cl. CPC C07K 16/2878; A61P 35/00; A61K 2039/505; C07K 2317/24; C07K 2317/33; C07K 2317/732; C07K 2317/76; C07K 2317/92; C07K 2317/94

(57) ABSTRACT

Provided are anti-TNFR2 (tumor necrosis factor receptor 2) antibodies, antigen-binding fragments, and the uses thereof.

### CLAIM OF PRIORITY

This application claims the benefit of international Application No. PCT/CN2021/140487, filed on Dec. 22, 2021. The entire contents of the foregoing are incorporated herein by reference.

### TECHNICAL FIELD

This disclosure relates to anti-TNFR2 (Tumor Necrosis Factor Receptor 2) antibodies and uses thereof.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million.

Recent clinical and commercial success of anticancer antibodies has created great interest in antibody-based therapeutics. There is a need to develop antibodies for use in various antibody-based therapeutics to treat cancers or auto-immune diseases.

### SUMMARY

This disclosure relates to anti-TNFR2 antibodies, antigen-binding fragment thereof, and the uses thereof.

In one aspect, the disclosure is related to an antibody or antigen-binding fragment thereof that binds to TNFR2, comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 6, 7, 8, respectively, and the selected VL CDRs 1, 2, 3 amino acid sequences are set forth in SEQ ID NOs: 9, 10, 11, respectively;
(2) the selected VH CDRs 1, 2, 3 amino acid sequences are set forth in SEQ ID NOs: 12, 13, 14, respectively, and the selected VL CDRs 1, 2, 3 amino acid sequences are set forth in SEQ ID NOs: 15, 16, 17, respectively;
(3) the selected VH CDRs 1, 2, 3 amino acid sequences are set forth in SEQ ID NOs: 18, 19, 20, respectively, and the selected VL CDRs 1, 2, 3 amino acid sequences are set forth in SEQ ID NOs: 21, 22, 23, respectively;
(4) the selected VH CDRs 1, 2, 3 amino acid sequences are set forth in SEQ ID NOs: 24, 25, 26, respectively, and the selected VL CDRs 1, 2, 3 amino acid sequences are set forth in SEQ ID NOs: 27, 28, 29, respectively;
(5) the selected VH CDRs 1, 2, 3 amino acid sequences are set forth in SEQ ID NOs: 30, 31, 32, respectively, and the selected VL CDRs 1, 2, 3 amino acid sequences are set forth in SEQ ID NOs: 33, 34, 35, respectively;
(6) the selected VH CDRs 1, 2, 3 amino acid sequences are set forth in SEQ ID NOs: 42, 43, 44, respectively, and the selected VL CDRs 1, 2, 3 amino acid sequences are set forth in SEQ ID NOs: 45, 46, 47, respectively;
(7) the selected VH CDRs 1, 2, 3 amino acid sequences are set forth in SEQ ID NOs: 48, 49, 50, respectively, and the selected VL CDRs 1, 2, 3 amino acid sequences are set forth in SEQ ID NOs: 51, 52, 53, respectively;
(8) the selected VH CDRs 1, 2, 3 amino acid sequences are set forth in SEQ ID NOs: 54, 55, 56, respectively, and the selected VL CDRs 1, 2, 3 amino acid sequences are set forth in SEQ ID NOs: 57, 58, 59, respectively;
(9) the selected VH CDRs 1, 2, 3 amino acid sequences are set forth in SEQ ID NOs: 60, 61, 62, respectively, and the selected VL CDRs 1, 2, 3 amino acid sequences are set forth in SEQ ID NOs: 63, 64, 65, respectively; or
(10) the selected VH CDRs 1, 2, 3 amino acid sequences are set forth in SEQ ID NOs: 66, 67, 68, respectively, and the selected VL CDRs 1, 2, 3 amino acid sequences are set forth in SEQ ID NOs: 69, 70, 71, respectively.

In some embodiments, the VH comprises CDRs 1, 2, 3 with the amino acid sequences set forth in SEQ ID NOs: 6, 7, and 8 respectively, and the VL comprises CDRs 1, 2, 3 with the amino acid sequences set forth in SEQ ID NOs: 9, 10, and 11, respectively according to Kabat numbering.

In some embodiments, the VH comprises CDRs 1, 2, 3 with the amino acid sequences set forth in SEQ ID NOs: 12, 13, and 14 respectively, and the VL comprises CDRs 1, 2, 3 with the amino acid sequences set forth in SEQ ID NOs: 15, 16, and 17, respectively according to Kabat numbering.

In some embodiments, the VH comprises CDRs 1, 2, 3 with the amino acid sequences set forth in SEQ ID NOs: 18, 19, and 20 respectively, and the VL comprises CDRs 1, 2, 3 with the amino acid sequences set forth in SEQ ID NOs: 21, 22, and 23, respectively according to Kabat numbering.

In some embodiments, the VH comprises CDRs 1, 2, 3 with the amino acid sequences set forth in SEQ ID NOs: 24, 25, and 26, respectively, and the VL comprises CDRs 1, 2, 3 with the amino acid sequences set forth in SEQ ID NOs: 27, 28, and 29, respectively according to Kabat numbering.

In some embodiments, the VH comprises CDRs 1, 2, 3 with the amino acid sequences set forth in SEQ ID NOs: 30, 31, and 32, respectively, and the VL comprises CDRs 1, 2, 3 with the amino acid sequences set forth in SEQ ID NOs: 33, 34, and 35, respectively according to Kabat numbering.

In some embodiments, the antibody or antigen-binding fragment specifically binds to human TNFR2.

In some embodiments, the antibody or antigen-binding fragment is a human antibody or antigen-binding fragment thereof (e.g., a human IgG1 antibody).

In some embodiments, the antibody or antigen-binding fragment is a single-chain variable fragment (scFV).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 7, and 8, respectively, and wherein the VH, when paired with a light chain variable region (VL) comprising the amino acid sequence set forth in SEQ ID NO: 37 binds to TNFR2;
(2) an immunoglobulin light chain or a fragment thereof comprising a VL comprising CDRs 1, 2, and 3 comprising the amino acid sequences set forth in SEQ ID NOs: 9, 10, and 11, respectively, and wherein the VL, when paired with a VH comprising the amino acid sequence set forth in SEQ ID NO: 36 binds to TNFR2;
(3) an immunoglobulin heavy chain or a fragment thereof comprising a heavy chain variable region (VH) comprising CDRs 1, 2, and 3 comprising the amino acid sequences set forth in SEQ ID NOs: 12, 13, and 14, respectively, and wherein the VH, when paired with a light chain variable region (VL) comprising the amino acid sequence set forth in SEQ ID NO: 39 binds to TNFR2;
(4) an immunoglobulin light chain or a fragment thereof comprising a VL comprising CDRs 1, 2, and 3 comprising the amino acid sequences set forth in SEQ ID NOs: 15, 16, and 17, respectively, and wherein the VL, when paired with a VH comprising the amino acid sequence set forth in SEQ ID NO: 38 binds to TNFR2;
(5) an immunoglobulin heavy chain or a fragment thereof comprising a heavy chain variable region (VH) comprising CDRs 1, 2, and 3 comprising the amino acid sequences set forth in SEQ ID NOs: 18, 19, and 20, respectively, and wherein the VH, when paired with a light chain variable region (VL) comprising the amino acid sequence set forth in SEQ ID NO: 41 binds to TNFR2;
(6) an immunoglobulin light chain or a fragment thereof comprising a VL comprising CDRs 1, 2, and 3 comprising the amino acid sequences set forth in SEQ ID NOs: 21, 22, and 23, respectively, and wherein the VL, when paired with a VH comprising the amino acid sequence set forth in SEQ ID NO: 40 binds to TNFR2;
(7) an immunoglobulin heavy chain or a fragment thereof comprising a heavy chain variable region (VH) comprising CDRs 1, 2, and 3 comprising the amino acid sequences set forth in SEQ ID NOs: 24, 25, and 26, respectively, and wherein the VH, when paired with a light chain variable region (VL) comprising the amino acid sequence set forth in SEQ ID NO: 73 binds to TNFR2;
(8) an immunoglobulin light chain or a fragment thereof comprising a VL comprising CDRs 1, 2, and 3 comprising the amino acid sequences set forth in SEQ ID NOs: 27, 28, and 29, respectively, and wherein the VL, when paired with a VH comprising the amino acid sequence set forth in SEQ ID NO: 72 binds to TNFR2;
(9) an immunoglobulin heavy chain or a fragment thereof comprising a heavy chain variable region (VH) comprising CDRs 1, 2, and 3 comprising the amino acid sequences set forth in SEQ ID NOs: 30, 31, and 32, respectively, and wherein the VH, when paired with a light chain variable region (VL) comprising the amino acid sequence set forth in SEQ ID NO: 75 binds to TNFR2; or
(10) an immunoglobulin light chain or a fragment thereof comprising a VL comprising CDRs 1, 2, and 3 comprising the amino acid sequences set forth in SEQ ID NOs: 33, 34, and 35, respectively, and wherein the VL, when paired with a VH comprising the amino acid sequence set forth in SEQ ID NO: 74 binds to TNFR2.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 6, 7, and 8, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 9, 10, and 11, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 12, 13, and 14, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 15, 16, and 17, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 18, 19, and 20, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 21, 22, and 23, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 24, 25, and 26, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 27, 28, and 29, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 30, 31, and 32, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 33, 34, and 35, respectively.

In some embodiments, the VH when paired with a VL specifically binds to human TNFR2, or the VL when paired with a VH specifically binds to human TNFR2.

In some embodiments, the immunoglobulin heavy chain or the fragment thereof is a human immunoglobulin heavy chain or a fragment thereof, and the immunoglobulin light chain or the fragment thereof is a human immunoglobulin light chain or a fragment thereof.

In some embodiments, the nucleic acid encodes a single-chain variable fragment (scFv).

In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VH region and the VL region that together bind to TNFR2.

In one aspect, the disclosure is related to a pair of vectors, wherein each vector comprises one of the nucleic acids described herein, wherein together the pair of vectors encodes the VH region and the VL region that together bind to TNFR2.

In one aspect, the disclosure is related to a cell comprising the vector described herein, or the pair of vectors described herein. In some embodiments, the cell is a CHO cell.

In one aspect, the disclosure is related to a cell comprising one or more of the nucleic acids described herein.

In one aspect, the disclosure is related to a cell comprising two of the nucleic acids described herein. In some embodiments, the two nucleic acids together encode the VH region and the VL region that together bind to TNFR2.

In one aspect, the disclosure is related to a method of producing an antibody or an antigen-binding fragment thereof, the method comprising (a) culturing the cell described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to TNFR2 comprising: a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 36, and the selected VL sequence is SEQ ID NO: 37;
(2) the selected VH sequence is SEQ ID NO: 38, and the selected VL sequence is SEQ ID NO: 39;
(3) the selected VH sequence is SEQ ID NO: 40, and the selected VL sequence is SEQ ID NO: 41;
(4) the selected VH sequence is SEQ ID NO: 72, and the selected VL sequence is SEQ ID NO: 73; or
(5) the selected VH sequence is SEQ ID NO: 74, and the selected VL sequence is SEQ ID NO: 75.

In some embodiments, the VH comprises the sequence of SEQ ID NO: 36 and the VL comprises the sequence of SEQ ID NO: 37.

In some embodiments, the VH comprises the sequence of SEQ ID NO: 72 and the VL comprises the sequence of SEQ ID NO: 73.

In some embodiments, the VH comprises the sequence of SEQ ID NO: 38 and the VL comprises the sequence of SEQ ID NO: 39.

In some embodiments, the VH comprises the sequence of SEQ ID NO: 40 and the VL comprises the sequence of SEQ ID NO: 41.

In some embodiments, the VH comprises the sequence of SEQ ID NO: 74 and the VL comprises the sequence of SEQ ID NO: 75.

In some embodiments, the antibody or antigen-binding fragment specifically binds to human TNFR2.

In some embodiments, the antibody or antigen-binding fragment is a human antibody or antigen-binding fragment thereof.

In some embodiments, the antibody or antigen-binding fragment is a single-chain variable fragment (scFV).

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to TNFR2 comprising: a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 36, and the selected VL sequence is SEQ ID NO: 37;
(2) the selected VH sequence is SEQ ID NO: 38, and the selected VL sequence is SEQ ID NO: 39;
(3) the selected VH sequence is SEQ ID NO: 40, and the selected VL sequence is SEQ ID NO: 41;
(4) the selected VH sequence is SEQ ID NO: 72, and the selected VL sequence is SEQ ID NO: 73; or
(5) the selected VH sequence is SEQ ID NO: 74, and the selected VL sequence is SEQ ID NO: 75.

In one aspect, the disclosure is related to an antibody-drug conjugate comprising the antibody or antigen-binding fragment thereof described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, or the antibody-drug conjugate described herein.

In some embodiments, the subject has colorectal cancer, ovarian cancer, acute myeloid leukemia, Lewis lung carcinoma, breast carcinoma, hepatocellular carcinoma and colon cancer, glioma. In some embodiments, the subject has renal cell carcinoma, multiple myeloma, colon cancer, ovarian cancer, glioma, or cutaneous T-cell lymphomas. In some embodiments, the cancer is colon cancer, glioma or ovarian cancer.

In one aspect, the disclosure is related to a method of decreasing the rate of tumor growth, the method comprising: contacting a tumor cell with an effective amount of a composition comprising an antibody or antigen-binding fragment thereof described herein, or the antibody-drug conjugate described herein.

In one aspect, the disclosure is related to a method of killing a tumor cell, the method comprising: contacting a tumor cell with an effective amount of a composition comprising the antibody or antigen-binding fragment thereof described herein, or the antibody-drug conjugate described herein.

In one aspect, the disclosure is related to a pharmaceutical composition comprising the antibody or antigen-binding fragment thereof described herein, and a pharmaceutically acceptable carrier.

In one aspect, the disclosure is related to a pharmaceutical composition comprising the antibody drug conjugate described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as head and neck, respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, glioma and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments. As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g.,TNFR2) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a TNFR2 molecule may be referred to as a TNFR2-specific antibody or an anti-TNFR2 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing the tumor size over time in mice injected with MC38 cancer cells and treated with PBS (phosphate-buffered saline) as a control (G1), anti-hTNFR2 antibodies BC-1F4-IgG1 (G2), BC-3B7-IgG1 (G3), BC-1F10-IgG(G4), 14-1B3-hHvKv-IgG1 (G5), BC-1A8-IgG1 (G6), BC-1C3-IgG1 (G7), 14-4A9-hHvKv-IgG1 (G8) and anti-mPD-1 (G9).

FIG. 2 is a graph showing the tumor size over time in mice injected with MC38 cancer cells and treated with PBS as a control (G1), antibodies BC-1A8-IgG1 (G2), BC-1F10-IgG1 (G3), BC-1F4-IgG1 (G4), anti-mPD-1 (G5), anti-mCTLA4 (G6).

FIG. 3 is a graph showing the tumor size over time in mice injected with MC38 cancer cells and treated with PBS as a control (G1), antibodies BC-1C3-lgG1 (G2), anti-mPD-1 (G3), anti-mCTLA4 (G4).

FIG. 4 is a graph showing the tumor size over time in mice injected with MC38 cancer cells and treated with PBS as a control (G1), antibodies BC-1A8-IgG1 (G2), BC-1C3-IgG1 (G3), BC-1F10-lgG1 (G4), BC-1F4-lgG1 (G5), BC-1B6-lgG1 (G6).

FIG. 5 is a graph showing the tumor size over time in mice injected with MC38 cancer cells and treated with PBS as a control (G1), BC-1A8-IgG1 (G2), BC-1C3-IgG1 (G3), BC-1F10-IgG1 (G4), BC-1F4-IgG1 (G5), BC-1B6-IgG1 (G6).

FIG. 6 is a graph showing the tumor size over time in hTNFα/hTNFR2 mice injected with MC38 cancer cells and treated with PBS as a control (G1), BC-1C3-lgG1 (G2), anti-mPD-1 (G3).

FIGS. 7A-7B show the testing results of the blood biochemical indicators (AST, ALT) in the peripheral blood of mice on the 5th day (D5) after group assignment.

FIG. 8 shows the epitope correlation among the anti-hTNFR2 antibodies.

FIG. 9 shows the cytotoxicity data for BC-1C3-IgG1, BC-1C3-IgG1-SI, BC-1C3-IgG1-LALA and human IgG1.

FIG. 10 is a drug concentration-time curve showing the change of the concentration of antibody drugs in the serum of the humanized TNFR2 mice after injection of different antibody drugs.

FIG. 11A shows the fluorescence signals indicating the activation of the reporter cells (Jurkat-GFP-TNFR2 cells).

FIG. 11B shows the fluorescence signals indicating the activation of the reporter cells (Jurkat-GFP-TNFR2 cells). TNFα is not displayed.

FIG. 12 shows the fluorescence signals indicating the activation of the reporter cells (Jurkat-GFP-TNFR2 cells).

FIGS. 13A-13B show the testing results of the blood biochemical indicators (AST, ALT) in the peripheral blood of mice.

FIG. 14 lists CDR sequences of anti-TNFR2 antibodies BC-1A8 (“1A8”), BC-1B6 (“1B6”), BC-1C3 (“1C3”), BC-1F4 (“1F4”), BC-1F10 (“1F10”) as defined by Kabat numbering scheme.

FIG. 15 lists CDR sequences of anti-TNFR2 antibodies BC-1A8 (“1A8”), BC-1B6 (“1B6”), BC-1C3 (“1C3”), BC-1F4 (“1F4”), BC-1F10 (“1F10”) as defined by Chothia numbering scheme.

FIG. 16 lists amino acid sequences of heavy chain variable regions and light chain variable regions of anti-TNFR2 antibodies (1A8, 1B6, 1C3, 1F4, and 1F10).

FIG. 17 lists certain relevant amino acid sequences.

FIG. 18 is a graph showing the tumor sizes over time in mice injected with MC38 cancer cells and treated with PBS (G1), BC-1C3-IgG1 (G2), anti-mPD-1 (G3), combination of BC-1C3-lgG1 and anti-mPD1 (G4), atezolizumab analog (G5) or combination of BC-1C3-lgG1 and atezolizumab analog (G6).

FIG. 19 is a graph showing the tumor sizes over time in mice injected with GL261 cancer cells and treated with PBS (G1), 1 mg/kg BC-1C3-IgG1 (G2), 3 mg/kg BC-1C3-IgG1 (G3), 10 mg/kg BC-1C3-IgG1 (G4) or anti-mPD-1 (G5).

FIG. 20 is a graph showing the tumor sizes over time in mice injected with MC38 cancer cells and treated with PBS (G1), BC-1C3-lgG1 (G2), BI-1808 analog (G3), h600-25-108 analog (G4), or HFB3-1hz6-hG1 analog (G5).

FIG. 21 is a graph showing the tumor sizes over time in mice injected with MC38 cancer cells and treated with PBS (G1), BC-1C3-lgG1 (G2), or h600-25-71 analog (G3).

FIG. 22 is a graph showing the proliferation of hCD8a+ T cells as determined by flow cytometry.

FIG. 23 is a graph showing the proliferation of hCD8a+ T cells as determined by flow cytometry.

FIG. 24A is a graph showing human IL12 release.

FIG. 24B is a graph showing human IFNγ release.

### DETAILED DESCRIPTION

Tumor necrosis factor (TNF) is generally considered a master proinflammatory cytokine (Al-Hatamleh et al. “A perspective review on the role of nanomedicine in the modulation of TNF-TNFR2 axis in breast cancer immunotherapy.” Journal of oncology 2019 (2019)). During inflammatory processes (including the cancer microenvironment), TNF is one inflammatory mediator that is produced secreted firstly. It fosters the generation of a cytokine cascade and promotes the production of other inflammatory mediators (e.g., transcription factors, interleukin (IL)-1, IL-6). There are two types of TNF receptors (TNFR1 and TNFR2) localized at the cellular surface. Experiments from inflammation-associated cancer revealed that TNFR2 is preferentially upregulated over TNFR1 and that treatment with the anti-TNF monoclonal antibody reduced the number and size of tumors. Therefore, TNF-TNFR2 axis was implicated in the suppression of immune response and affects tumor progression and metastasis.

The present disclosure provides examples of antibodies, antigen-binding fragment thereof, that bind to TNFR2 (tumor necrosis factor receptor 2).

### TNFR2 and Cancer

T cells have been an important focus of cancer immunotherapy due to their capacity to directly kill tumor cells if they are CD8+ cytotoxic T lymphocytes (CTLs), also known as CD8+ effector T cells (Teff). However, Treg cells can suppress Teff cells and thereby deter the proper host immune response to eliminate a tumor. Thus, inhibition of suppressive Treg cells and the simultaneous activation of cytotoxic CD8+ Teff can be a potential strategy to treat cancer (Vanamee, Éva S., et al. “TNFR2: a novel target for cancer immunotherapy.” Trends in molecular medicine 23.11 (2017): 1037-1046)

TNFR2 is a member of the TNFR superfamily (TNFRSF) and is activated by TNF. It is a cell-surface receptor that regulates cell survival and proliferation and targeting this receptor has emerged as a potential next-generation cancer therapeutic approach. Certain human tumor cells can aberrantly express TNFR2 and tumor infiltrates are dominated by highly suppressive TNFR2+ Treg cells.

TNFR2 is predominately expressed in cells of the immune system, especially regulatory T (Treg) cells, and by endothelial cells, and preferentially binds transmembrane TNF (tmTNF). TNFR1 and TNFR2 are single-pass transmembrane glycoproteins with 28% homology mainly in the extracellular domain, that is comprised of four cysteine-rich motif. However, the intracellular domains of the TNF receptors are largely unrelated, lacking homologous sequences, suggesting that different signaling functions originate from the two distinct receptors. TNFR1 contains an intracellular death domain (DD) that binds TNFR1-associated death domain protein (TRADD) of Fas-associated death domain (FADD), primarily involved in signaling for cell death. While TNFR2 does not contain a cytoplasmic DD, it interacts with TNF-associated factor 2 (TRAF2), and mainly yields cell survival. The signaling circuitry of TNFR2 differs from that of the other TNFR. TNFR1 contains an intracellular death domain and can activate either apoptotic or inflammatory pathways, while TNFR2 binds TNF receptor-associated factors (TRAFs) and can activate both the canonical and the non-canonical NF-KB pathway to control cell survival and proliferation in both humans and mice.

TNFR2 has 461 amino acids, amino acids 1-22 being signal peptide, amino acids 23-257 being the extracellular domain, amino acids 258-287 being the transmembrane domain, and amino acids 288-461 being the cytoplasmic domain with a TRAF2 binding site. TRAF2 can bind TRAF1, TRAF3, inhibitor of apoptosis protein 1 (cIAP1) and inhibitor of apoptosis protein 2 (cIAP2).

Antagonistic anti-TNFR2 antibodies can block ligand binding and lock membrane receptors in a resting (non-signaling), antiparallel dimer arrangement, while agonistic, cross-linking antibodies can stabilize parallel TNF-TNFR2 complexes, i.e., provide a structural stabilization of the active signaling network. In addition, it is well recognized now that TNFR2 contributes to the stabilization of the CD4+Foxp3+ Treg phenotype in the inflammatory environment.

A detailed description of TNFR2 and its function can be found, e.g., in Al-Hatamleh, et al. “A perspective review on the role of nanomedicine in the modulation of TNF-TNFR2 axis in breast cancer immunotherapy.” Journal of oncology 2019 (2019); Vanamee, et al. “TNFR2: a novel target for cancer immunotherapy.” Trends in molecular medicine 23.11 (2017): 1037-1046; Orti-Casañ, et al. “Targeting TNFR2 as a novel therapeutic strategy for Alzheimer's disease.” Frontiers in neuroscience 13 (2019): 49; Chen, et al. “Interaction of TNF with TNF receptor type 2 promotes expansion and function of mouse CD4+ CD25+ T regulatory cells.” The Journal of Immunology 179.1 (2007): 154-161; each of which is incorporated by reference in its entirety.

The present disclosure provides anti-TNFR2 antibodies, antigen-binding fragments thereof, and methods of using these anti-TNFR2 antibodies and antigen-binding fragments to inhibit tumor growth and to treat various diseases, including e.g., cancer.

### Anti-TNFR2 Antibodies and Antigen-Binding Fragments

The disclosure provides antibodies and antigen-binding fragments thereof that specifically bind to TNFR2 (e.g., human TNFR2). The antibodies and antigen-binding fragments described herein are capable of binding to TNFR2. In some embodiments, these antibodies can block TNFR2 signaling pathway thus increase immune response. In some embodiments, these antibodies can initiate complement-dependent cytotoxicity (CMC) or antibody-dependent cellular cytotoxicity (ADCC).

The disclosure provides e.g., anti-TNFR2 antibodies BC-1A8 (“1A8”), BC-1B6 (“1B6”), BC-1C3 (“1C3”), BC-1F4 (“1F4”), BC-1F10 (“1F10”), BC-3B7 (“3B7”), the modified antibodies thereof, including e.g., chimeric antibodies, humanized antibodies, and human antibodies.

The CDR sequences for 1A8, and 1A8 derived antibodies (e.g., humanized antibodies) include CDRs of the heavy chain variable domain, SEQ ID NOs: 6, 7, 8, and CDRs of the light chain variable domain, SEQ ID NOs: 9, 10, 11 as defined by Kabat numbering. The CDRs can also be defined by Chothia system. Under the Chothia numbering, the CDR sequences of the heavy chain variable domain are set forth in SEQ ID NOs: 42, 43, 44, and CDR sequences of the light chain variable domain are set forth in SEQ ID NOs: 45, 46, 47.

The CDR sequences for 1B6, and 1B6 derived antibodies include CDRs of the heavy chain variable domain, SEQ ID NOs: 12, 13, 14, and CDRs of the light chain variable domain, SEQ ID NOs: 15, 16, 17, as defined by Kabat numbering. Under Chothia numbering, the CDR sequences of the heavy chain variable domain are set forth in SEQ ID NOs: 48, 49, 50, and CDRs of the light chain variable domain are set forth in SEQ ID NOs: 51, 52, 53.

The CDR sequences for 1C3, and 1C3 derived antibodies include CDRs of the heavy chain variable domain, SEQ ID NOs: 18, 19, 20, and CDRs of the light chain variable domain, SEQ ID NOs: 21, 22, 23, as defined by Kabat numbering. Under Chothia numbering, the CDR sequences of the heavy chain variable domain are set forth in SEQ ID NOs: 54, 55, 56, and CDRs of the light chain variable domain are set forth in SEQ ID NOs: 57, 58, 59.

The CDR sequences for 1F4, and 1F4 derived antibodies include CDRs of the heavy chain variable domain, SEQ ID NOs: 24, 25, 26, and CDRs of the light chain variable domain, SEQ ID NOs: 27, 28, 29, as defined by Kabat numbering. Under Chothia numbering, the CDR sequences of the heavy chain variable domain are set forth in SEQ ID NOs: 60, 61, 62, and CDRs of the light chain variable domain are set forth in SEQ ID NOs: 63, 64, 65.

The CDR sequences for 1F10, and 1F10 derived antibodies include CDRs of the heavy chain variable domain, SEQ ID NOs: 30, 31, 32, and CDRs of the light chain variable domain, SEQ ID NOs: 33, 34, 35, as defined by Kabat numbering. Under Chothia numbering, the CDR sequences of the heavy chain variable domain are set forth in SEQ ID NOs: 66, 67, 68, and CDRs of the light chain variable domain are set forth in SEQ ID NOs: 69, 70, 71.

The amino acid sequence for the heavy chain variable region of 1A8 antibody is set forth in SEQ ID NO: 36. The amino acid sequence for the light chain variable region of 1A8 antibody is set forth in SEQ ID NO: 37.

The amino acid sequence for the heavy chain variable region of 1B6 antibody is set forth in SEQ ID NO: 38. The amino acid sequence for the light chain variable region of 1B6 antibody is set forth in SEQ ID NO: 39.

The amino acid sequence for the heavy chain variable region of 1C3 antibody is set forth in SEQ ID NO: 40. The amino acid sequence for the light chain variable region of 1C3 antibody is set forth in SEQ ID NO: 41.

The amino acid sequence for the heavy chain variable region of 1F4 antibody is set forth in SEQ ID NO: 72. The amino acid sequence for the light chain variable region of 1F4 antibody is set forth in SEQ ID NO: 73.

The amino acid sequence for the heavy chain variable region of 1F10 antibody is set forth in SEQ ID NO: 74. The amino acid sequence for the light chain variable region of 1F10 antibody is set forth in SEQ ID NO: 75.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 36, 38, 40, 72 or 74. In some embodiments, the light chain variable region is at least 80%, 85%, 90%, 91%, 92%, 93%, 94%, 95%, 96%, 97%, 98%, or 99% identical to SEQ ID NO: 37, 39, 41, 73 or 75. The heavy chain variable region sequence can be paired with the corresponding light chain variable region sequence, and together they bind to TNFR2.

Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, IGHD, and IGHJ genes), and/or human kappa chain immunoglobulin locus sequences (e.g., recombination of human IGKV and IGKJ genes).

Furthermore, in some embodiments, the antibodies or antigen-binding fragments thereof described herein can also contain one, two, or three heavy chain variable region CDRs selected from the group of SEQ ID NOs: 6-8, SEQ ID NOs: 12-14, SEQ ID NOs: 18-20, SEQ ID NOS: 24-26 and SEQ ID NOs: 30-32 (Kabat numbering); and/or one, two, or three light chain variable region CDRs selected from the group of SEQ ID NOs: 9-11, SEQ ID NOs: 15-17, SEQ ID NOs: 21-23, SEQ ID NOs: 27-29 and SEQ ID NOs: 33-35 (Kabat numbering).

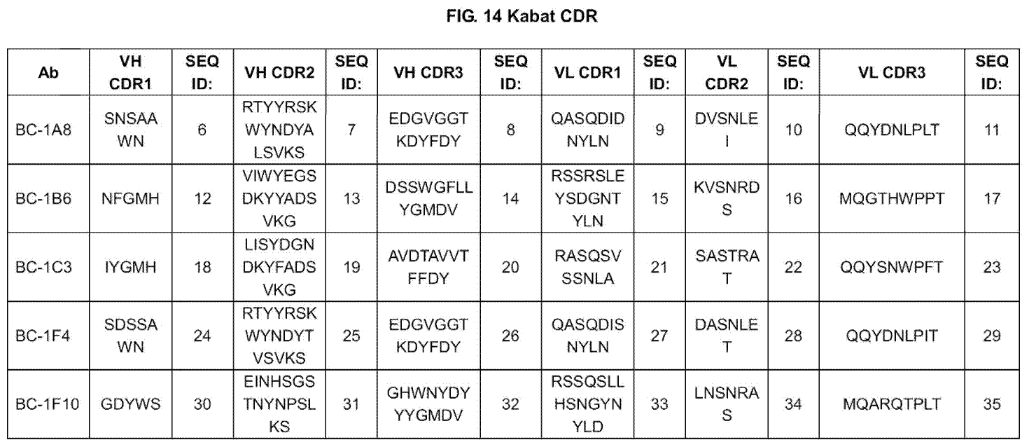

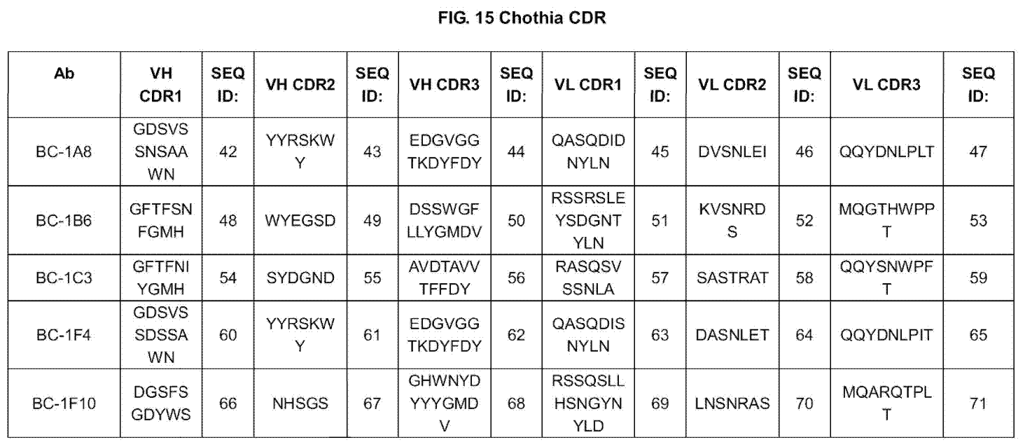

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 14 (Kabat CDR) and FIG. 15 (Chothia CDR).

In some embodiments, the antibody or an antigen-binding fragment described herein can contain a heavy chain variable domain containing one, two, or three of the CDRs of SEQ ID NO: 6 with zero, one or two amino acid insertions, deletions, or substitutions; SEQ ID NO: 7 with zero, one or two amino acid insertions, deletions, or substitutions; SEQ ID NO: 8 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 12 with zero, one or two amino acid insertions, deletions, or substitutions; SEQ ID NO: 13 with zero, one or two amino acid insertions, deletions, or substitutions; SEQ ID NO: 14 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 18 with zero, one or two amino acid insertions, deletions, or substitutions; SEQ ID NO: 19 with zero, one or two amino acid insertions, deletions, or substitutions; SEQ ID NO: 20 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 24 with zero, one or two amino acid insertions, deletions, or substitutions; SEQ ID NO: 25 with zero, one or two amino acid insertions, deletions, or substitutions; SEQ ID NO: 26 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 30 with zero, one or two amino acid insertions, deletions, or substitutions; SEQ ID NO: 31 with zero, one or two amino acid insertions, deletions, or substitutions; SEQ ID NO: 32 with zero, one or two amino acid insertions, deletions, or substitutions.

In some embodiments, the antibody or an antigen-binding fragment described herein can contain a light chain variable domain containing one, two, or three of the CDRs of SEQ ID NO: 9 with zero, one or two amino acid insertions, deletions, or substitutions; SEQ ID NO: 10 with zero, one or two amino acid insertions, deletions, or substitutions; SEQ ID NO: 11 with zero, one or two amino acid insertions, deletions, or substitutions.

In some embodiments, the antibody or an antigen-binding fragment described herein can contain a light chain variable domain containing one, two, or three of the CDRs of SEQ ID NO: 15 with zero, one or two amino acid insertions, deletions, or substitutions; SEQ ID NO: 16 with zero, one or two amino acid insertions, deletions, or substitutions; SEQ ID NO: 17 with zero, one or two amino acid insertions, deletions, or substitutions.

In some embodiments, the antibody or an antigen-binding fragment described herein can contain a light chain variable domain containing one, two, or three of the CDRs of SEQ ID NO: 21 with zero, one or two amino acid insertions, deletions, or substitutions; SEQ ID NO: 22 with zero, one or two amino acid insertions, deletions, or substitutions; SEQ ID NO: 23 with zero, one or two amino acid insertions, deletions, or substitutions.

In some embodiments, the antibody or an antigen-binding fragment described herein can contain a light chain variable domain containing one, two, or three of the CDRs of SEQ ID NO: 27 with zero, one or two amino acid insertions, deletions, or substitutions; SEQ ID NO: 28 with zero, one or two amino acid insertions, deletions, or substitutions; SEQ ID NO: 29 with zero, one or two amino acid insertions, deletions, or substitutions.

In some embodiments, the antibody or an antigen-binding fragment described herein can contain a light chain variable domain containing one, two, or three of the CDRs of SEQ ID NO: 33 with zero, one or two amino acid insertions, deletions, or substitutions; SEQ ID NO: 34 with zero, one or two amino acid insertions, deletions, or substitutions; SEQ ID NO: 35 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to TNFR2. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 36, and the selected VL sequence is SEQ ID NO: 37. In some embodiments, the selected VH sequence is SEQ ID NO: 38 and the selected VL sequence is SEQ ID NO: 39. In some embodiments, the selected VH sequence is SEQ ID NO: 40 and the selected VL sequence is SEQ ID NO: 41. In some embodiments, the selected VH sequence is SEQ ID NO: 72 and the selected VL sequence is SEQ ID NO. 73. In some embodiments, the selected VH sequence is SEQ ID NO: 74 and the selected VL sequence is SEQ ID NO: 75.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

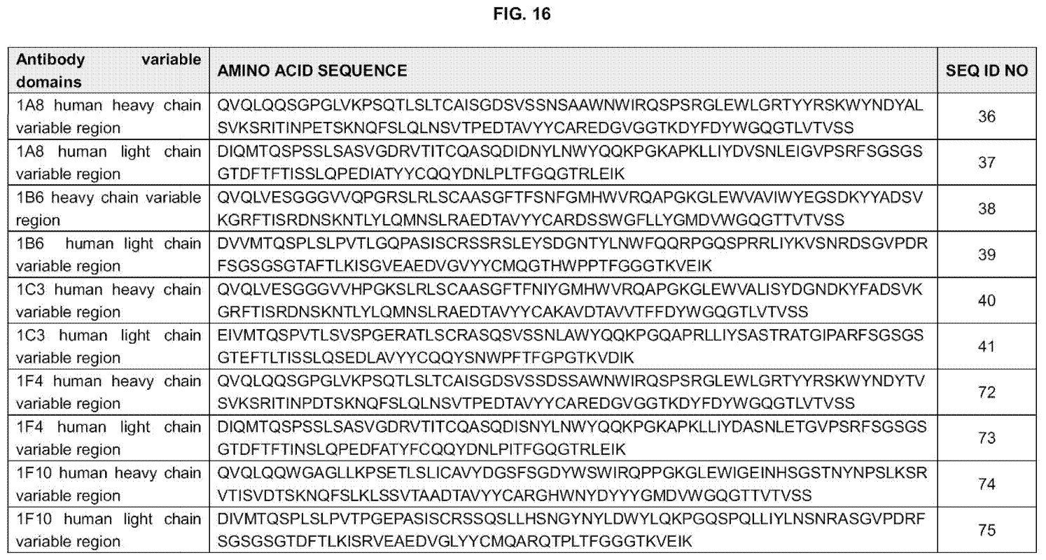

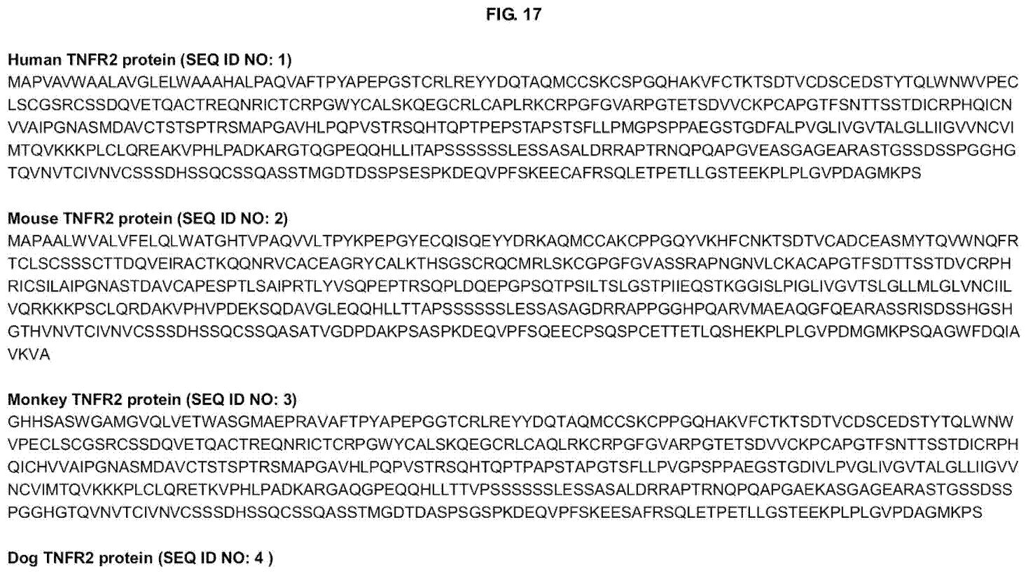

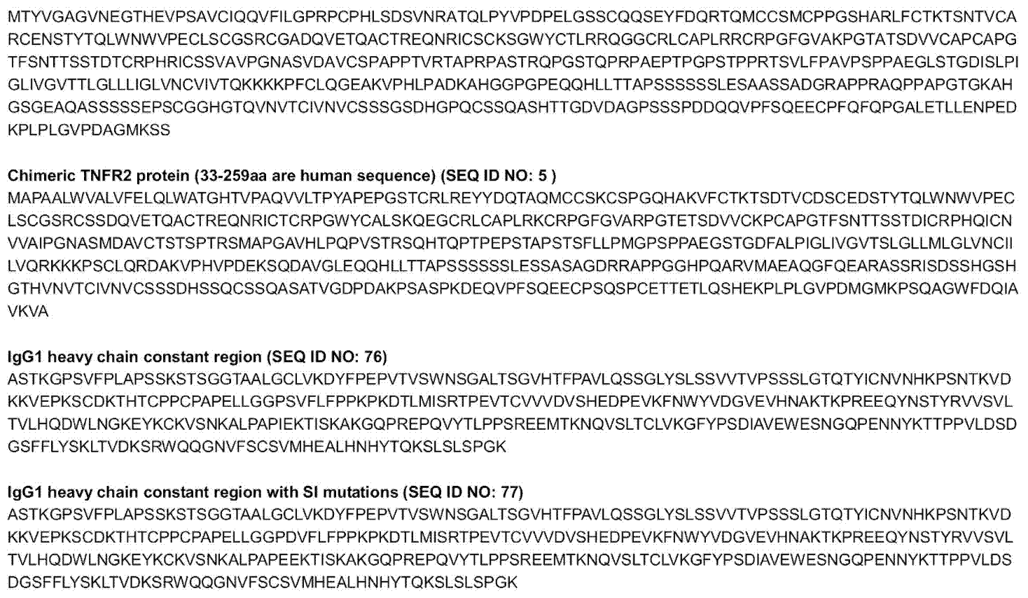

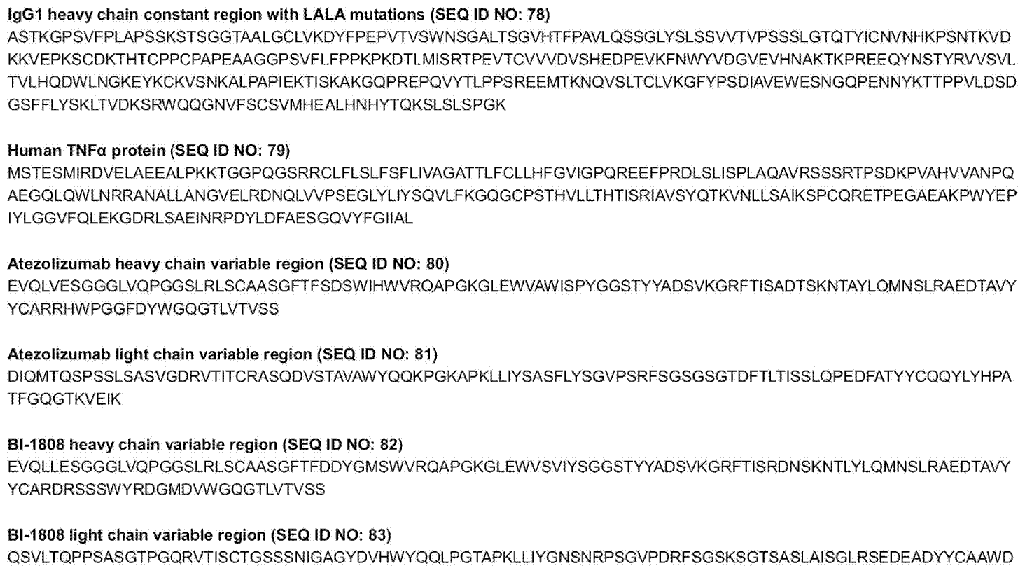

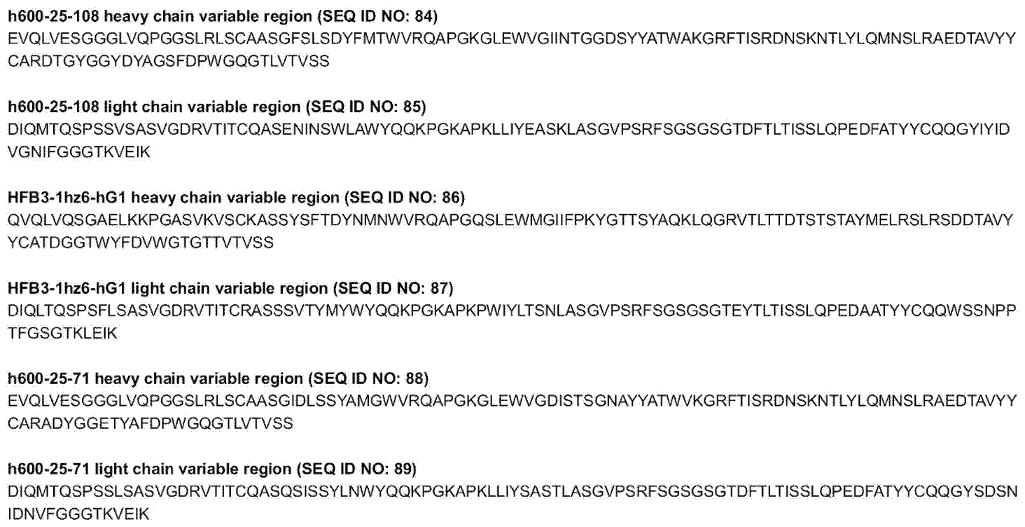

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 14 or FIG. 15, or have sequences as shown in FIG. 16. When the polypeptides are paired with corresponding polypeptide (e.g., a corresponding heavy chain variable region or a corresponding light chain variable region), the paired polypeptides bind to TNFR2.

The anti-TNFR2 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to TNFR2 will retain an ability to bind to TNFR2. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides various antibodies and antigen-binding fragments thereof derived from anti-TNFR2 antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007), each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain. In some embodiments, the scFV has two heavy chain variable domains, and two light chain variable domains.

### Antibody Characteristics

The antibodies or antigen-binding fragments thereof described herein can block the binding between TNFR2 and TNFR2 ligands. In some embodiments, by binding to TNFR2, the antibody can inhibit TNFR2 signaling pathway. In some embodiments, the antibody can upregulate immune response. In some embodiments, the antibody can reduce tumor volume in an animal with tumors expressing TNFR2.

In some implementations, the antibody (or antigen-binding fragments thereof) specifically binds to TNFR2 (human TNFR2, mouse TNFR2, monkey TNFR2, dog TNFR2, chimeric TNFR2) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD-koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human TNFR2 (SEQ ID NO: 1), mouse TNFR2 (e.g., SEQ ID NO: 2), monkey TNFR2 (e.g., SEQ ID NO: 3), dog TNFR2 (SEQ ID NO: 4), and/or chimeric TNFR2 (SEQ ID NO: 5). In some embodiments, the antibody does not bind to monkey TNFR2, dog TNFR2, chimeric TNFR2, and/or mouse TNFR2.

TNFR2 has four cysteine rich domains (CRD). CRD1 is the 39aa-76aa of SEQ ID NO: 1, CRD2 is the 77aa-118aa of SEQ ID NO: 1, CRD3 is the 119aa-162aa of SEQ ID NO: 1, and CRD4 is the 168aa-196aa of SEQ ID NO: 1. In some embodiments, the antibody (or antigen-binding fragments thereof) specifically binds to CRD1, CRD2, CRD3, and/or CRD4. In some embodiments, the epitope is located at the junction of CRD3 and CRD4.

In some embodiments, the antibody or antigen-binding fragment thereof described herein can block the inhibition of the proliferation of CD8+T cells by Treg cells.

In some embodiments, the antibody or antigen-binding fragment thereof described herein can promote the proliferation of CD8+ T cells. In some embodiments, the antibody or antigen-binding fragment thereof described herein can promote the proliferation of CD8+ T cells by more than 10%, more than 15%, more than 20%, more than 25%, more than 30%, more than 40%, more than 50%, more than 60%, more than 70% or more than 80%.

In some embodiments, the antibody or antigen-binding fragment thereof described herein can enhance the cytokine release by CD8+ T cells. In some embodiments, the antibody or antigen-binding fragment thereof described herein can enhance the release of IL-2 by CD8+ T cells. In some embodiments, the antibody or antigen-binding fragment thereof described herein can enhance the release of IL-2 by CD8+ T cells by more than 10%, more than 15%, more than 20%, more than 25%, more than 30%, more than 40%, more than 50%, more than 60%, more than 70% or more than 80%. In some embodiments, the antibody or antigen-binding fragment thereof described herein can enhance the release of IFN-γ by CD8+ T cells. In some embodiments, the antibody or antigen-binding fragment thereof described herein can enhance the release of IFN-γ by CD8+ T cells by more than 10%, more than 15%, more than 20%, more than 25%, more than 30%, more than 40%, more than 50%, more than 60%, more than 70% or more than 80%.

In some embodiments, the antibody or antigen-binding fragment thereof described herein has a tumor growth inhibition percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI_{TV}% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI_{TV}%) is calculated using the following formula:

$${TGI}_{TV} ( \% ) = [ 1 - ( Ti - T 0 ) / ( Vi - V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen-binding fragments thereof as described herein have cytotoxicity, e.g., for cells expressing TNFR2. The methods of measuring cytotoxicity are known in the art. In some embodiments, the cytotoxicity is calculated by the following formula:

$$\% Cytotoxicity = ( \frac{( Experimental - Spontaneous ) - ( Effector - Spontaneous ) - ( Target - Spontaneous )}{Target Maximum - Spontaneous *} ) \times 100 \%$$

Wherein Experimental refers to Absorbance value of experimental well (effector cell+target cell+test product); Effector refers to the absorbance value of effector cells (only effector cells). Target refers to the absorbance value of target cells (only target cells). Spontaneous refers to the autofluorescence of the cell culture media (only cell culture media, no effector cells, target cells) absorbance value. Target Maximum refers to the highest absorbance value of the target cells (target cell+lysate). Spontaneous* refers to absorbance value of cell culture media volume control well (only medium+lysate). EC50 can also be calculated. In some embodiments, EC50 is less than 200, 150, 100, 90, 80, 70, 60, 50, 40, 30, 20, or 10 μg/mL.

In some embodiments, the antibodies or antigen-binding fragments thereof as described herein are TNFR2 antagonist. In some embodiments, the antibodies or antigen binding fragments decrease TNFR2 signal transduction in a target cell (e.g., T cell such as Treg) that expresses TNFR2.

In some embodiments, the antibodies or antigen-binding fragments thereof as described herein are not toxic. In some embodiments, no significant differences in body weight can be observed between treatment group and control group e.g., at 0.3 mg/kg, 1 mg/kg, 10 mg/kg, or 25 mg/kg.

In some embodiments, the antibodies or antigen binding fragments can bind to tumor cells that express TNFR2. In some embodiments, the antibodies or antigen binding fragments can induce complement-dependent cytotoxicity (CMC) and/or antibody dependent cellular cytoxicity (ADCC), and kill the tumor cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments can induce complement mediated cytotoxicity (CMC).

In some embodiments, the Fc region is human IgG1, human IgG2, human IgG3, or human IgG4. In some embodiments, the antibody is a human IgG1 antibody.

In some embodiments, the antibodies or antigen binding fragments do not have a functional Fc region. For example, the antibodies or antigen binding fragments are Fab, Fab′, F(ab′)2, and Fv fragments. In some embodiments, the Fc region has LALA mutations (L234A and L235A mutations in EU numbering), or LALA-PG mutations (L234A, L235A, P329G mutations in EU numbering).

In some embodiments, the Fc have a SI mutation (S239D and 1332E mutations in EU numbering).

### Methods of Making Anti-TNFR2 Antibodies

An isolated fragment of human TNFR2 (e.g., extracellular region) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of TNFR2 and encompasses an epitope of the protein such that an antibody raised against the peptide forms a specific immune complex with the protein. As described above, the full length sequence of human TNFR2 is known in the art (SEQ ID NO: 1). In some embodiments, an Fc-tagged human TNFR2 protein (the Fc fusion protein contains human TNFR2 extracellular domain, positions 23-257 of SEQ ID NO: 1) is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human TNFR2). The preparation can further include an adjuvant, such as Freund's complete or incomplete adjuvant, or a similar immunostimulatory agent.

Polyclonal antibodies can be prepared as described above by immunizing a suitable subject with a TNFR2 polypeptide, or an antigenic peptide thereof (e.g., part of TNFR2, such as the extracellular region) as an immunogen. The antibody titer in the immunized subject can be monitored over time by standard techniques, such as with an enzyme-linked immunosorbent assay (ELISA) using the immobilized TNFR2 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds), John Wiley & Sons, Inc, New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., TNFR2. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al.

“Replacing the complementarity-determining regions in a human antibody with those from a mouse.” Nature 321.6069 (1986): 522; Riechmann et al. “Reshaping human antibodies for therapy.” Nature 332.6162 (1988): 323; Dall'Acqua et al. “Antibody humanization by framework shuffling.” Methods 36.1 (2005): 43-60; each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al. “A humanized CD18antibody can block function without cell destruction.” The Journal of Immunology 151.4 (1993): 2296-2308; Chothia, et al., “Canonical structures for the hypervariable regions of immunoglobulins.” Journal of molecular biology 196.4 (1987): 901-917).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-TNFR2 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab mice can be found in PCT/CN2020/075698, which is incorporated herein by reference in its entirety. The antibodies generated by the mice have a full human VH, a full human VL, and mouse constant regions. In some embodiments, the human VH and human VL is linked to a human IgG constant regions (e.g., IgG1, IgG2, IgG3, and IgG4). In some embodiments, the constant region has a sequence that is at least at least 80%, 85%, 90%, 91%, 92%, 93%, 94%, 95%, 96%, 97%, 98%, 99% or 100% identical to SEQ ID NO: 76, 77, 78, or 90.

Identity or homology with respect to an original sequence is usually the percentage of amino acid residues present within the candidate sequence that are identical with a sequence present within the human, humanized, or chimeric anti-TNFR2 antibody or fragment, after aligning the sequences and introducing gaps, if necessary, to achieve the maximum percent sequence identity, and not considering any conservative substitutions as part of the sequence identity.

Additional modifications to the anti-TNFR2 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-TNFR2 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, neurologic cancer, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, glioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

In some aspects, the disclosure relates to a method of treating an autoimmune disease or inflammation, the method comprising administering to the subject an effective amount of a composition comprising the antibody or antigen-binding fragment thereof as described herein or the antibody-drug conjugate as described herein.

In one aspect, the disclosure provides methods for treating, preventing, or reducing the risk of developing disorders associated with an abnormal or unwanted immune response, e.g., an autoimmune disorder, e.g., by affecting the function of Treg. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-TNFR2 antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has Crohn's disease, ulcerative colitis or type 1 diabetes. In some embodiments, the antibodies or antigen binding fragments can be used to treat inflammation.

In some embodiments, the antibody or antigen binding fragment thereof is a TNFR2 agonist.

In some aspects, the disclosure relates to a method of inhibiting immune response in a subject, the method comprising administering to the subject an effective amount of a composition comprising the antibody or antigen-binding fragment thereof as described herein or the antibody-drug conjugate as described herein. In some embodiments, the subject has an autoimmune disease.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal.

A typical daily dosage of an effective amount of an antibody is 0.01 mg/kg to 100 mg/kg (mg per kg of patient weight). In some embodiments, the dosage can be less than 100 mg/kg, 50 mg/kg, 40 mg/kg, 30 mg/kg, 25 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.3 mg/kg, or 0.1 mg/kg. In some embodiments, the dosage can be greater than 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.3 mg/kg, 0.1 mg/kg, 0.05 mg/kg, or 0.01 mg/kg. In some embodiments, the dosage is about 50 mg/kg, 40 mg/kg, 30 mg/kg, 25 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, IL-1, HMGB1, an IL-10 antagonist, an IL-4 antagonist, an IL-6 antagonist (e.g., IL-6 receptor), an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, an anti-GITR antibody, anti-TIM-3 antibody or anti-CD40 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 0.3 mg/kg to about 25 mg/kg, about 1 mg/kg to about 10 mg/kg; or about 1 mg/kg to about 5 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-hTNFR2 Antibodies

To generate antibodies against human TNFR2 (TNFR2; SEQ ID NO: 1), RenMab mice were immunized with human TNFR2. Anti-TNFR2 antibodies were made by the methods as described below.

RenMab mice have both a humanized heavy chain immunoglobulin locus and a humanized kappa chain immunoglobulin locus. The heavy chain immunoglobulin locus is a region on the chromosome that contains genes for the heavy chains of antibodies. The locus includes IGHV (variable), IGHD (diversity), IGHJ (joining), and heavy chain constant domain genes. The kappa chain immunoglobulin locus is a region on the chromosome that contains genes that encode the light chains of antibodies (kappa chain). The kappa chain immunoglobulin locus includes IGKV (variable), IGKJ (joining), and light chain constant domain genes. A detailed description regarding RenMab mice can be found in PCT/CN2020/075698, which is incorporated herein by reference in its entirety.

### Immunization of Mice

RenMab mice were immunized with Fc-tagged human TNFR2 proteins (the Fc fusion protein contains human TNFR2 extracellular domain, positions 23aa-257aa of SEQ ID NO: 1). The Fc-tagged human TNFR2 proteins were emulsified with adjuvant and injected at four positions on the back of the mice. For the first subcutaneous (s.c.) injection, the diluted antigen was emulsified with Complete Freund's Adjuvant (CFA) in equal volume. In the following subcutaneous injections, the protein was emulsified with Incomplete Freund's Adjuvant (IFA) in equal volume. At least four injections were performed with at least 14 days between every two injections. Seven days after the third injection or the booster immunization, blood (serum) was collected and analyzed for antibody titer using Fluorescence-Activated Cell Sorting (FACS).

In another experiment, several mice were immunized by injecting the expression plasmid encoding human TNFR2 into the mice. The plasmids encoding the antigen were injected into the tibialis anterior muscle (intramuscular injection; i.m. injection). At least four injections were performed with at least 14 days between every two injections. Blood (serum) was collected seven days after the last immunization and the serum was tested for antibody titer by ELISA.

Procedures to enhance immunization were also performed at least fourteen days after immunization (either by injecting the plasmid or by injecting the proteins). CHO cells that express TNFR2 antigen on the surface were intravenously injected into the mice through tail veins. Immune system organs (e.g., bone marrow, lymph node, spleen, etc.) was then collected four days after the injection.

The spleen cells were collected and fused with mouse myeloma cells to retain their viability and form a hybridoma cell line. The hybridoma cells were screened and selected to identify cell lines that produced TNFR2 specific antibodies. Using this technique and the aforementioned immunogen, several anti-TNFR2 chimeric antibodies (i.e., antibodies with human variable domains and mouse constant domains) were obtained. Specifically, after the impulse immunization, the mouse immune organs were harvested and the plasma cells were separated by magnetic beads. The hybridoma fusion technology was used to screen out the monoclonal hybridoma cells that secrete antigen-specific monoclonal antibodies. The antibody light and heavy chain V region sequences was obtained by reverse transcription and PCR sequencing in the selected monoclonal hybridoma cells. The antibody light and heavy chain V region sequences were constructed into the antibody expression vector and verified by the Expi CHO-STM CELLs expression system. Cells were transfected in a 24-well system, and antibodies were collected on the 3rd day in the supernatant. FACS was used to verify the specificity of the binding between the antibody and TNFR2. Using this technique, several anti-TNFR2 chimeric antibodies (i.e., antibodies with human variable domains and mouse constant domains) were obtained. The constant domains in these antibodies can be easily replaced to obtain fully human anti-TNFR2 antibodies (anti-hTNFR2 antibody). The exemplary fully human antibodies obtained by this method are named as follows: 14-1B3-hHvKv (“14-1B3” or “1B3”) and 14-4A9-hHvKv (“14-4A9” or “4A9”)), etc. Using 14-4A9-hHvKv as an example, when the antibody VH\VL is connected to a different subtype like the IgG1 subtype, the antibody is named 14-4A9-hHvKv-IgG1.

The antigen-positive B cells were also directly isolated from the immunized mice without fusion with myeloma cells. The anti-TNFR2 antibodies were further isolated from the antigen-positive B cells. The light and heavy chain V region sequences of the antibody were directly obtained from the antigen-positive B cells. For example, single cell technology (e.g., Beacon® Optofluidic System, Berkeley Lights Inc.) was used to screen and find plasma cells that secrete antigen-specific monoclonal antibodies. The antibody V region sequences were obtained using reverse transcription and PCR sequencing. The antibodies were expressed. FACS was used to verify the binding between the antibody and TNFR2. Exemplary antibodies obtained by this method include: BC-1A8 (“1A8”), BC-1B6 (“1B6”), BC-1C3 (“1C3”), BC-1F4 (“1F4”), BC-1F10 (“1F10”), BC-3B7 (“3B7”). Take BC-1F4 as an example, when the antibody VH\VL is connected to a different subtype like the IgG1 subtype, the antibody is named BC-1F4-IgG1. Examples of other subtypes are as follows: BC-1F4-IgG1-SI, BC-1F4-IgG1-LALA.

The heavy chain and light chain CDR1, CDR2, and CDR3 amino acid sequences for 1A8 were shown in SEQ ID NOs: 6-11 (Kabat numbering) or SEQ ID NOs: 42-47 (Chothia numbering). The antibody's human heavy chain variable region and human light chain variable region were shown in SEQ ID NO: 36 or SEQ ID NO: 37.

The heavy chain and light chain CDR1, CDR2, and CDR3 amino acid sequences for 1B6 were shown in SEQ ID NOs: 12-17 (Kabat numbering) or SEQ ID NOs: 48-53 (Chothia numbering). The antibody's human heavy chain variable region and human light chain variable region were shown in SEQ ID NO: 38 or SEQ ID NO: 39.

The heavy chain and light chain CDR1, CDR2, and CDR3 amino acid sequences for 1C3 were shown in SEQ ID NOs: 18-23 (Kabat numbering) or SEQ ID NOs: 54-59 (Chothia numbering). The antibody's human heavy chain variable region and human light chain variable region were shown in SEQ ID NO: 40 or SEQ ID NO: 41.

The heavy chain and light chain CDR1, CDR2, and CDR3 amino acid sequences for 1F4 were shown in SEQ ID NOs: 24-29 (Kabat numbering) or SEQ ID NOs: 60-65 (Chothia numbering). The antibody's human heavy chain variable region and human light chain variable region were shown in SEQ ID NO: 72 or SEQ ID NO: 73.

The heavy chain and light chain CDR1, CDR2, and CDR3 amino acid sequences for 1F10 were shown in SEQ ID NOs: 30-35 (Kabat numbering) or SEQ ID NOs: 66-71 (Chothia numbering). The antibody's human heavy chain variable region and human light chain variable region were shown in SEQ ID NO: 74 or SEQ ID NO: 75.

### Antibody Preparation

The positive antibody sequences in the sequence verification stage were subjected to plasmid extraction and transfection into a 25mL system. The expression supernatants were collected after 10-12 days of cell culture and subjected to affinity chromatography. The antibody samples obtained were used in the following in vitro testing and screening.

### Example 2. In Vitro Testing of Anti-TNFR2 Antibodies

### Blocking the Binding of Human TNFR2 and TNFα

Blocking assays were performed to determine whether the anti-TNFR2 antibodies can block the binding between TNFR2 and its ligand hTNFα.

Specifically, 30 μl CHO cells (1×10⁵ cells) transiently transfected with human TNF Receptor 2 (TNFR2) were added to each well in a plate. The purified antibodies were titrated to final concentrations of 10, 2.5, 0.625, 0.1565, 0.039 μg/mL. The titrated antibodies were added to each well at 30 μl per well at 4° C. and incubated for 30 minutes.

30 μl of Biotin-hTNFα (Acro Biosystems, Cat #: TNA-H82E1) was added to each well (with a final concentration of 0.5 μg/mL in each well). The cells with Biotin-hTNFα and the antibodies were incubated at 4° C. for 30 minutes.

After two washes with phosphate-buffered saline (PBS), 50 μl of PE labeled anti-human IgG Fc antibody (PE Anti-human IgG Fc, Jackson ImmunoResearch, Cat #: 109-115-098) at 1:100 dilution and Alexa Fluor® 647-labeled Streptavidin (AF647 Streptavidin, Jackson ImmunoResearch Cat #: 016-600-084) at 1:500 dilution were added into each well, and incubated for 15 minutes at 4° C., followed by a PBS wash. The signals for AF647 and PE were determined by flow cytometry (Thermo Attune NX).

Table 1 below shows the percentage of tested cells that had Streptavidin signals in the flow cytometry analysis. An antibody has blocking affinity (indicating strong binding affinity) if the percentage of tested cells that had Streptavidin signals (AF647) increases while the antibody concentration decreases. Based on the data, BC-1A8-IgG1, BC-1F4-IgG1, BC-3B7-IgG1 and BC-1F10-IgG1 had strong blocking effects. But BC-1B6-IgG1, BC-1C3-IgG1, 14-1B3-hHvKv-IgG1 and 14-4A9-hHvKv-IgG1 cannot effectively block the binding between TNFR2 and TNFα.

TABLE 1

| | 10 | 2.5 | 0.625 | 0.1565 | 0.039 | |
|---|---|---|---|---|---|---|
| Antibody | μg/mL | μg/mL | μg/mL | μg/mL | μg/mL | Blocking |
| BC-1A8-IgG1 | 12.0% | 29.9% | 58.6% | 67.4% | 62.8% | Yes |
| BC-1B6-IgG1 | 75.1% | 87.2% | 80.4% | 82.7% | 78.9% | No |
| BC-1C3-IgG1 | 71.1% | 81.5% | 77.3% | 74.3% | 77.0% | No |
| BC-1F4-IgG1 | 18.5% | 59.5% | 61.9% | 76.2% | 84.6% | Yes |
| BC-1F10-IgG1 | 51.9% | 54.5% | 60.2% | 55.2% | 59.0% | Yes |
| BC-3B7-IgG1 | 19.4% | 46.6% | 69.7% | 70.1% | 64.5% | Yes |
| 14-1B3-hHvKv-IgG1 | 66.6% | 65.1% | 62.5% | 63.2% | 63.8% | No |
| 14-4A9-hHvKv-IgG1 | 66.1% | 65.7% | 60.7% | 63.2% | 63.0% | No |

### Binding Affinity of Anti-TNFR2 Antibodies to Human TNFR2 and TNFR1

The binding affinity of the anti-TNFR2 antibodies to human TNFR2 and TNFR1 (Tumor necrosis factor receptor 1) were measured using surface plasmon resonance (SPR) using Biacore (Biacore, INC, Piscataway NJ) 8K biosensor equipped with pre-immobilized Protein A sensor chips.

Purified anti-TNFR2 antibodies were diluted to 1 μg/mL and then injected into the Biacore 8K biosensor at 10 μL/min for about 50 seconds to achieve a desired protein density (e.g., about 50 response units (RU)). His-tagged human TNFR1 (Human TNFR1/CD120a/TNFRSF1A Protein, His Tag, Beijing Acrobiosystems CO.LTD., Cat #: TN1-5222) or TNFR2 (Human TNFR2/CD120b/TNFRSFIB Protein, His Tag, Beijing Acrobiosystems CO.LTD., Cat #: TN2-5227) at concentrations of 200, 100, 50, 25, 6.25 or 1.56 nM were then injected at 30 μL/min for 120 seconds. Dissociation was monitored for 600 seconds. The chip was regenerated after the last injection of each titration with Glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in Table 2 below.

TABLE 2

| | | Association | Dissociation | |
|---|---|---|---|---|
| Anti-TNFR2 | | rate | rate | Affinity |
| antibodies | Analyte | kon (1/Ms) | koff (1/s) | KD (M) |
| BC-1A8-IgG1 | hTNFR2-His | 9.24E+05 | 1.26E−03 | 1.36E−09 |
| BC-1A8-IgG1 | hTNFR1-His | Negative | | |
| BC-1B6-IgG1 | hTNFR2-His | 1.66E+06 | 6.44E−03 | 3.89E−09 |
| BC-1B6-IgG1 | hTNFR1-His | Negative | | |
| BC-1C3-IgG1 | hTNFR2-His | 1.21E+05 | 1.15E−03 | 9.50E−09 |
| BC-1C3-IgG1 | hTNFR1-His | Negative | | |
| BC-1F4-IgG1 | hTNFR2-His | 7.18E+05 | 1.59E−03 | 2.21E−09 |
| BC-1F4-IgG1 | hTNFR1-His | Negative | | |
| BC-1F10-IgG1 | hTNFR2-His | 9.15E+04 | 4.67E−04 | 5.10E−09 |
| BC-1F10-IgG1 | hTNFR1-His | Negative | | |
| 14-1B3-hHvKv-IgG1 | hTNFR2-His | 4.85E+04 | 4.22E−04 | 8.70E−09 |
| 14-1B3-hHvKv-IgG1 | hTNFR1-His | Negative | | |
| 14-4A9-hHvKv-IgG1 | hTNFR2-His | 3.95E+04 | 1.82E−03 | 4.61E−08 |
| 14-4A9-hHvKv-IgG1 | hTNFR 1-His | Negative | | |

The results show that these human antibodies have very high binding affinity with human TNFR2. None of the eight antibodies (BC-1A8-IgG1, BC-1B6-IgG1, BC-1C3-IgG1, BC-1F4-IgG1, BC-1F10-IgG1, 14-1B3-hHvKv-IgG1 and 14-4A9-hHvKv-IgG1) can bind to TNFR1.

### Binding Affinity of Anti-TNFR2 Antibodies to Monkey TNFR2

Similar to the aforementioned binding affinity experiment, the binding affinities of the anti-TNFR2 antibodies BC-1B6-IgG1, BC-1C3-IgG1 and BC-1F4-IgG1 to His-tagged monkey (Crab-eating monkey) TNFR2 (fTNFR2-His, Sino Biological, Cat #: 90102-C08H) were measured. The results are summarized in Table 3 below, which show that all three anti-TNFR2 antibodies can bind monkey TNFR2 with good binding affinities.

TABLE 3

| Anti-TNFR2 | | | | Affinity |
|---|---|---|---|---|
| antibodies | Analyte | Kon(1/Ms) | koff (1/s) | KD (M) |
| BC-1B6-IgG1 | fTNFR2-His | 3.13E+05 | 5.47E−03 | 1.75E−08 |
| BC-1C3-IgG1 | fTNFR2-His | 1.87E+05 | 2.21E−02 | 1.18E−07 |
| BC-1F4-IgG1 | fTNFR2-His | 5.41E+05 | 1.09E−02 | 2.01E−08 |

### Cross-Reactivity of Anti-TNFR2 Antibodies Against Mouse, Canine, and Monkey TNFR2

In each experiment, the CHO cells were transfected with EGFP and human TNFR2 (TNFR2, SEQ ID NO: 1), mouse TNFR2 (mTNFR2, SEQ ID NO: 2), fTNFR2, or dog (Canine) TNFR2 (dTNFR2, SEQ ID NO: 4).

30 μl CHO cells (1×10⁵ cells) were added to each well. 30 μl purified anti-TNFR2 antibodies (10 μg/mL) (as listed in Table 4) were added to each well and were incubated at 4° C. for 30 minutes.

After two washes with PBS (1600 rmp, 6 min), 50 μl of Alexa Fluor labeled anti-human IgG Fc antibody (BC-1A8-IgG1, BC-1B6-IgG1, BC-1C3-IgG1, BC-1F4-IgG1, BC-1F10-IgG, BC-3B7-IgG1, 14-1B3-hHvKv-IgG1, 14-4A9-hHvKv-IgG1) was added into each well 1:500 dilution, incubated at 4° C. for 15 minutes, followed by a PBS wash (1200 rmp, 5 min). The signals for AF647 were detected by flow cytometry.

The cross reactivity for the tested antibodies with human (TNFR2), mouse (mTNFR2), monkey (fTNFR2) and dog (dTNFR2) TNFR2 is summarized in the table below.

TABLE 4

| | TNFR2 | mTNFR2 | fTNFR2 | dTNFR2 |
|---|---|---|---|---|
| Protein | (human) | (Mouse) | (Monkey) | (Dog) |
| BC-1A8-IgG1 | Yes | No | Yes | No |
| BC-1B6-IgG1 | Yes | No | Yes | No |
| BC-1C3-IgG1 | Yes | No | Yes | No |
| BC-1F4-IgG1 | Yes | No | Yes | No |
| BC-1F10-IgG1 | Yes | No | Yes | No |
| BC-3B7-IgG1 | Yes | No | Yes | No |
| 14-1B3-hHvKv-IgG1 | Yes | No | Yes | No |
| 14-4A9-hHvKv-IgG1 | Yes | No | Yes | No |

### Epitope Correlation Analysis of Purified Anti-hTNFR2 Antibodies

Relative positions of target protein epitope between a pair of purified anti-TNFR2 monoclonal antibodies were analyzed through a surface plasmon resonance (SPR) competition experiment. A total of 5 monoclonal antibodies were used to study the binding inhibition (blocking) effect of each antibody on another antibody: BC-1A8-IgG1, BC-1F4-IgG1, BC-3B7-IgG1, BC-1C3-IgG1 and BC-1F10-IgG1. HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH 7.4) was used as the running buffer throughout the experiment. Anti-His antibodies were fixed on the surface of a Series S sensor Chip CM5 by amino group coupling to generate an anti-His chip (i.e., CM5-Anti-His-Channel 1,8-Chip). Then, 1M ethanolamine, pH 8.5 was injected to block the remaining active carboxyl groups on the chip surface, followed by equilibration using the HBS-EP+ buffer for 2 hours. Recombinant human TNFR2 proteins with His-tag (1 μg/mL) were injected into the Biacore 8K biosensor at 10 μL/min for 50 seconds and captured on the anti-His chip to achieve a desired protein density (i.e., 200 RU). A pair of antibodies (200 nM each) was continuously injected at 30 μL/min onto the chip. The first injected antibody (analyte 1) had a binding time of 250 seconds, and then the second antibody (analyte 2) was injected with a binding time of 250 seconds. After injection of the antibodies in each analysis cycle, the chip was regenerated twice with a glycine buffer (pH 1.7; 30 μL/min for 30 seconds). Each pair of monoclonal antibodies was subjected to the same experimental steps to obtain the binding inhibition data when each monoclonal antibody was paired with another antibody.

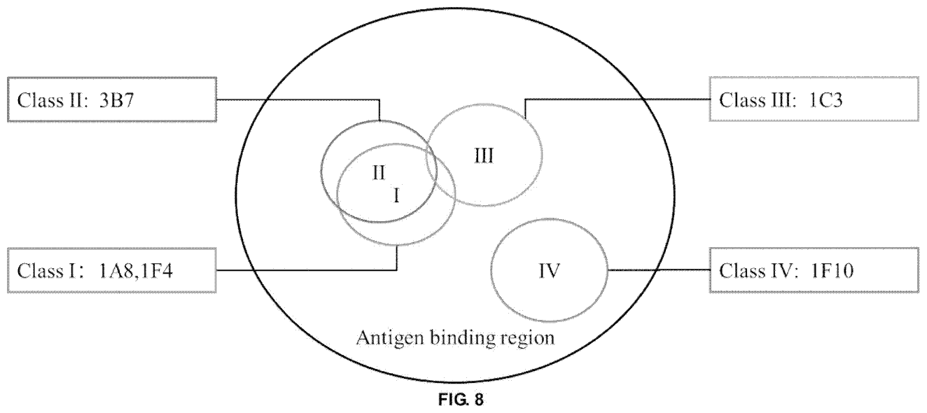

The binding value of each antibody was obtained using Biacore Insight Evaluation Software. To quantify the interference of one antibody binding to another, a binding ratio was calculated to compare each pair of antibodies. The binding ratio is defined as the binding value of the second antibody (analyte 2), divided by the binding value of the first antibody (analyte 1). A statistical software was also used for cluster analysis. Epitope correlation was analyzed and the 5 anti-hTNFR2 antibodies were categorized into 4 epitope clusters (FIG. 8). In summary, 1A8 and 1F4 shared the same or overlapping epitopes. 3B7, 1C3 and F10 do not exhibit epitope correlation with the other antibodies.

TNFR2 has four cysteine rich domains (CRD). In another experiment, FACS was used to detect the binding of anti-hTNFR2 antibodies to TNFR2 proteins with different structural domains. Secondary antibody (Alexa Fluor® 647 AffiniPure F(ab′) 2 Fragment Goat Anti-Human IgG, Fcγ fragment specific, Jackson ImmunoResearch Laboratories, Inc., Cat #: 151524) was used. The fluorescence-labeled secondary antibody can attach to the Fc region of the anti-hTNFR2 antibodies so that the anti-hTNFR2 antibodies that are bound to the ΔCRD cells can be detected by FACS. Specifically, ΔCRD1-TNFR2 protein (with CRD1 deleteion), ΔCRD2-TNFR2 protein (with CRD2 deleteion), ΔCRD3-TNFR2 protein (with CRD3 deleteion) or ΔCRD4-TNFR2 protein (with CRD4 deleteion) were expressed in CHO-S cells respectively, and then the binding between the cells and different anti-hTNFR2 antibodies was detected. The TNFR2 protein is shown in SEQ ID NO:1, CRD1 is the 39aa-76aa of the sequence, CRD2 is the 77aa-118aa of the sequence, CRD3 is the 119aa-162aa of the sequence, and CRD4 is the 168aa-196aa of the sequence. The results of the flow cytometry experiment are shown in Table 5 below.

The results showed that 1A8 and 1F4 did not bind to TNFR2 lacking CRD3, and the positive rate of binding to TNFR2 lacking CRD4 also decreased significantly, indicating that 1A8 and 1F4 have similar binding epitopes to human TNFR2 and the epitopes are likely to be located in CRD3 (class I epitopes). 1B6 and 1C3 did not bind to TNFR2 lacking CRD3 or CRD4, indicating that 1B6 and 1C3 have similar binding epitopes to human TNFR2, and the eptiopes are likely to be located at the junction of CRD3 and CRD4 (class III epitopes). The positive rate of binding between 1F10 and TNFR2 lacking CRD3 or CRD4 decreased significantly, indicating that the binding epitope for 1F10 is different from class I epitopes and class III epitopes (belonging to class IV epitopes). The results were consistent with the SPR epitope characterization.

TABLE 5

| | CHO- | CHO- | CHO- | CHO- | |
|---|---|---|---|---|---|
| | SΔCRD1- | SΔCRD2- | SΔCRD3- | SΔCRD4- | CHO- |
| Protein | TNFR2 | TNFR2 | TNFR2 | TNFR2 | S |
| BC-1A8-IgG1 | 64.6% | 71.2% | 0.52% | 29.4% | 1.44% |
| BC-1B6-IgG1- | 66.5% | 75.6% | 0.44% | 0.83% | 1.67% |
| LALA | | | | | |
| BC-1C3-IgG1 | 72.6% | 80.3% | 0.81% | 1.85% | 1.64% |
| BC-1F4-IgG1 | 53.1% | 59.1% | 0.47% | 21.3% | 1.44% |
| BC-1F10-IgG1 | 55.3% | 65.7% | 23.1% | 38.3% | 0.95% |
| Secondary | 10.2% | 7.33% | 0.62% | 1.02% | 1.64% |
| antibody | | | | | |

### In Vitro ADCC Detection Experiment

Experiments were performed to evaluate ADCC effects of anti-TNFR2 antibodies. In the experiment, BC-1A8-IgG1, BC-1C3-IgG1, BC-1F4-IgG1, BC-1F10-IgG1, BC-1B6-IgG1, and isotype control Human IgG1 (Crown Bioscience Inc., C0001-3) were used.

Lactate dehydrogenase (LDH) is a cytoplasmic enzyme found in many different cell types and is released into the cell culture medium after plasma membrane damage. CyQUANT LDH Cytotoxicity Detection Kit (Invitrogen, Cat #: C20301) was used to accurately and quantitatively measure extracellular LDH to evaluate the ADCC effect of antibodies.

Anti-TNFR2 antibodies were serially diluted (10-fold) with the highest concentration of 100 μg/mL. Target cells (MC38 cells over expressing human TNFR2) were seeded in a 96-well plate (cell density 2×10⁴ cells/well, 100 μL) and incubated at 37° C. for 3-4 hours. Effector cells (peripheral blood mononuclear cell (PBMC)) was resuscitated (cell density 2×10⁴ cells/well). The same volume (100 μL) of effector cells was added to each well of the 96-well plate together with 10 μl antibody. The above 96-well plate was incubated at 37° C. overnight. The 490 nm and 680 nm absorbance values were detected by a microplate reader and were used to calculate killing of target cells by each group of antibodies (Cytotoxicity %). EC50 value was calculated using nonlinear fitting using the antibody concentration as the horizontal axis and cytotoxicity as the vertical axis.

$$\% Cytotoxicity = ( \frac{( Experimental - Spontaneous ) - ( Effector - Spontaneous ) - ( Target - Spontaneous )}{Target Maximum - Spontaneous *} ) \times 100 \%$$

In the formula above, Experimental refers to Absorbance value of experimental well; Effector refers to the absorbance value of effector cells. Target refers to the absorbance value of target cells. Spontaneous refers to the autofluorescence of the cell culture media absorbance value. Target Maximum refers to the highest absorbance value of the target cells. Spontaneous* refers to absorbance value of cell culture media volume control well.

The EC50 results are shown in Table 6. Compared with the isotype control hIgG1, killing of target cells (Cytotoxicity %) by anti-TNFR2 antibodies (BC-1A8-IgG1, BC-1C3-IgG1, BC-1F4-IgG1, BC-1F10-IgG1 or BC-1B6-IgG1) increased with increasing dose of these antibodies, indicating that BC-1A8-IgG1, BC-1C3-IgG1, BC-1F4-IgG1, BC-1F10-IgG1 and BC-1B6-IgG1 all have ADCC activity.

TABLE 6

| Protein | ADCC | EC50 value (ng/mL) |
|---|---|---|
| BC-1A8-IgG1 | Yes | 86.41 |
| BC-1B6-IgG1 | Yes | 16.00 |
| BC-1C3-IgG1 | Yes | 96.94 |
| BC-1F4-IgG1 | Yes | 40.25 |
| BC-1F10-IgG1 | Yes | 161.50 |
| hIgG1 | No | — |

In another similar experiment, Target cells (MC38 cells over expressing human TNFR2) and Effector cells (FcR-TANK (CD16a-158V) Cell Line, ImmuneOnco Biopharmaceuticals (Shanghai) Co., Ltd.) were performed to evaluate ADCC effects of anti-TNFR2 antibodies (BC-1C3-IgG1, BC-1C3-IgG1-SI and BC-1C3-IgG1-LALA)

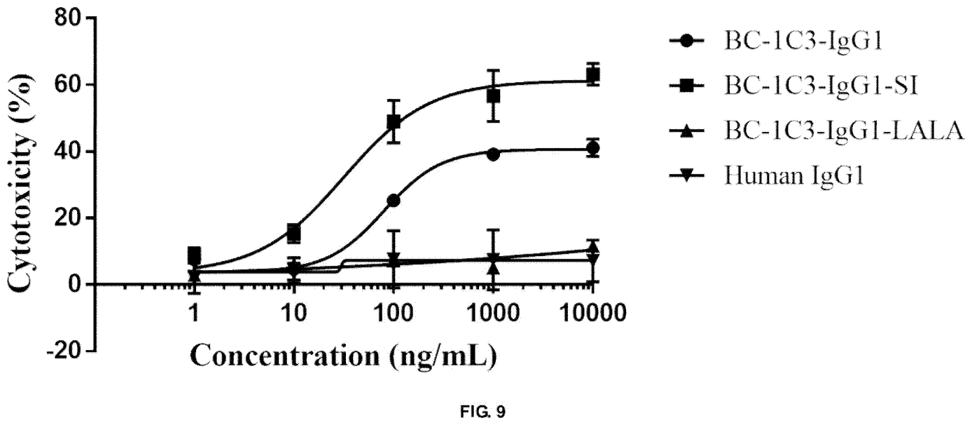

Cytotoxicity data are shown in FIG. 9. The EC50 results are shown in Table 7. Compared with the isotype control hlgG1, killing of target cells (Cytotoxicity %) by anti-hTNFR2 antibodies (BC-1C3-IgG1 and BC-1C3-IgG1-SI) increased with increasing dose of these antibodies, indicating that BC-1C3-IgG1-SI all have stronger ADCC activity.

TABLE 7

| Protein | ADCC | EC50 value (ng/mL) |
|---|---|---|
| BC-1C3-IgG1 | Yes | 80.69 |
| BC-1C3-IgG1-SI | Yes | 31.92 |
| BC-1C3-IgG1-LALA | No | — |
| hIgG1 | No | — |

### Example 3. In Vivo Testing of Anti-hTNFR2 Antibodies

In order to test the antibodies in vivo and to predict the effects of these antibodies in human, a humanized TNFR2 mouse model was generated. The humanized TNFR2 mouse model was engineered to express a chimeric TNFR2 protein (SEQ ID NO: 5) wherein the extracellular region of the mouse TNFR2 protein was replaced with the corresponding human TNFR2 extracellular region. The amino acid residues 33-260 of mouse TNFR2 (SEQ ID NO: 2) were replaced by amino acid residues 33-259 of human TNFR2 (SEQ ID NO: 1).

The humanized TNFR2 mouse model (e.g., B-TNFR2 mice) provides a new tool for testing new therapeutic treatments in a clinical setting by significantly decreasing the difference between clinical outcome in human and in ordinary mice expressing mouse TNFR2. A detailed description regarding humanized TNFR2 mouse models can be found in PCT/CN2020/113618; which is incorporated herein by reference in its entirety.

The anti-hTNFR2 antibodies were tested in TNFR2 humanized mice (B-TNFR2) to demonstrate their effect on tumor growth in vivo.

B-TNFR2 Mice With MC38, 10 mg/kg In Vivo Efficacy

MC-38 tumor cells (colon adenocarcinoma cell) were injected subcutaneously in B-TNFR2 mice. When the tumors in the mice reached a volume of 100 mm³-150 mm³, the mice were randomly placed into different groups based on the volume of the tumor (six mice in each group).

The mice were then injected with PBS as a control (G1), anti-hTNFR2 antibodies BC-1F4-IgG1 (G2), BC-3B7-IgG1 (G3), BC-1F10-IgG (G4), 14-1B3-hHvKv-IgG1 (G5), BC-1A8-IgG1 (G6), BC-1C3-IgG1 (G7), 14-4A9-hHvKv-IgG1 (G8) and anti-mPD-1 (G9) (BIO X CELL, INC., Cat #: BE0146). Anti-mPD-1, which had been shown to be efficacious in mouse, was used as a positive control. The antibodies were given on the first day and the fourth day of each week by intraperitoneal injection at 10 mg/kg for 3 weeks (6 injections in total).

The weight of the mice was monitored during the entire treatment period. The weight of mice in different groups all increased, with no statistically significant differences (P>0.05). On the day of group assignment (Day 0; “D0”), the average weight of each group was in the range of 20.3 g-21.4 g. At the end of the experiment (28 days after group assignment, D28) the average weight of each group was in the range of 22.7 g-24.7 g, the weight changes being in the range of 107.4%-116.5%. The results showed that the anti-hTNFR2 antibodies were well tolerated and not toxic to the mice.

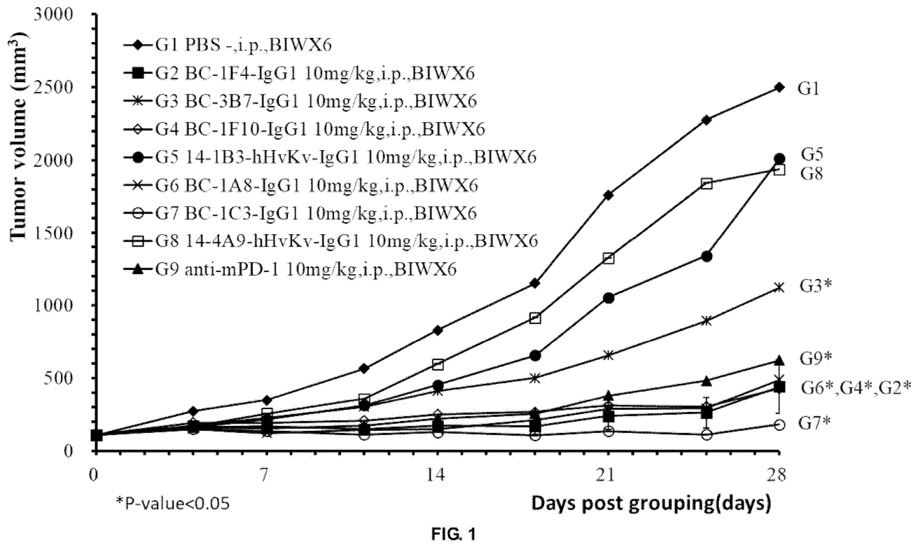

The tumor size (FIG. 1) showed significant differences in groups treated with the anti-hTNFR2 antibodies. The TGI_{TV}% on Day 28 (28 days after group assignment) for each treatment group is shown in Table 8 below. The positive control (anti-mPD-1, G9) showed a good therapeutic effect (TGI_{TV}%=78.5%) under the above-mentioned dosage and frequency. Multiple antibodies including BC-1F4-IgG1 (G2), BC-1F10-IgG1 (G4), BC-1A8-IgG1 (G6), BC-1C3-IgG1 (G7) showed good therapeutic effect (TGI_{TV}% >60%). The TGI_{TV}% of the TNFR2 antibodies, except for the G3 and G5, were greater than 80%. This TGI_{TV}% was better than the positive control group (G19/anti-mPD-1 TGI_{TV}%=78.5%).

TABLE 8

| | | | | | | | | | P value | |
|---|---|---|---|---|---|---|---|---|---|---|
| | | Tumor volume(mm³) | | | | | Tumor | TGI_{TV} | Body | Tumor |
| | | D 0 | D 11 | D 21 | D 28 | Survival | free | % | weight | Volume |
| Control | G1 | 109 ± 5 | 565 ± 115 | 1761 ± 310 | 2497 ± 231 | 4/6 | 0 | n.a. | n.a. | n.a. |
| Treat | G2 | 109 ± 6 | 148 ± 30 | 238 ± 93 | 441 ± 185 | 6/6 | 2 | 86.1% | 0.374 | 6.09E−05 |
| | G3 | 109 ± 5 | 302 ± 114 | 655 ± 237 | 1124 ± 442 | 6/6 | 1 | 57.5% | 0.433 | 0.030 |
| | G4 | 109 ± 5 | 208 ± 33 | 310 ± 71 | 429 ± 131 | 6/6 | 1 | 86.6% | 0.420 | 1.91E−05 |
| | G5 | 109 ± 6 | 312 ± 49 | 1055 ± 152 | 2012 ± 153 | 6/6 | 0 | 20.3% | 0.384 | 0.105 |
| | G6 | 109 ± 5 | 142 ± 20 | 290 ± 64 | 491 ± 113 | 6/6 | 0 | 84.0% | 0.474 | 1.74E−05 |
| | G7 | 109 ± 5 | 112 ± 13 | 136 ± 43 | 181 ± 70 | 6/6 | 1 | 97.0% | 0.310 | 2.57E−06 |
| | G8 | 109 ± 4 | 358 ± 70 | 1327 ± 282 | 1936 ± 391 | 4/6 | 0 | 23.5% | 0.660 | 0.253 |
| | G9 | 109 ± 6 | 171 ± 34 | 379 ± 102 | 622 ± 164 | 6/6 | 0 | 78.5% | 0.526 | 8.05E−05 |

B-TNFR2 Mice With MC38, 1 mg/kg In Vivo Efficacy

Similar to the aforementioned in vivo drug efficacy experiments, after constructing the tumor model, the mice were injected with PBS as a control (G1), anti-hTNFR2 antibodies BC-1A8-IgG1 (G2), BC-1F10-IgG1 (G3), BC-1F4-IgG1 (G4), anti-mPD-1 (G5), anti-mCTLA4 (G6) (BIO X CELL, INC., Cat #: BE0164). The antibodies were given on the first day and the fourth day of each week by intraperitoneal injection at 1 mg/kg for 3 weeks (6 injections in total).

The weight of the mice was monitored throughout the entire experiment. The weight of mice in different groups all increased and there was no significant difference among the groups (P>0.05). At the time of group assignment (D0), the average weight of each group was in the range of 20.4 g-20.8 g. At the end of the experiment (24 days after group assignment, D24), the average weight of each group was in the range of 21.6 g-23.7 g, and the weight change was in the range of 102.5%-114.7%. Similar to the previous experiments, the results showed that the anti-hTNFR2 antibodies were well tolerated and not toxic to the mice.

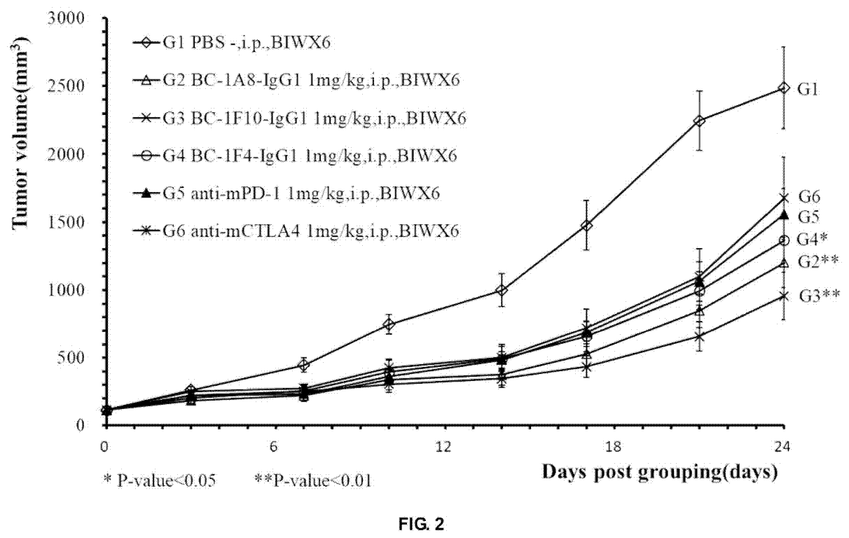

The tumor size showed significant difference in groups treated with the anti-TNFR2 antibodies (FIG. 2). Particularly, the tumor size in G3, G4 and G5 was significantly smaller than G1 (G3: P=0.005, G7: P=0.001, G8: P-0.011). The TGI_{TV}% at Day 24 (24 days after group assignment) for each treatment group was also calculated as shown in Table 9 below. G2, G3 and G4 showed better tumor inhibition (TGI_{TV}%) comparing to the positive control (G5, G6).

TABLE 9

| | | | | | | | | P value | |
|---|---|---|---|---|---|---|---|---|---|
| | | Tumor volume(mm³) | | | | | TGI_{TV} | Body | Tumor |
| | | D 0 | D 7 | D 17 | D 24 | Survival | % | weight | Volume |
| Control | G1 | 114 ± 5 | 447 ± 52 | 1476 ± 182 | 2486 ± 302 | 4/6 | n.a. | n.a. | n.a. |
| Treat | G2 | 114 ± 7 | 221 ± 45 | 528 ± 96 | 1203 ± 186 | 6/6 | 54.1% | 0.241 | 0.005 |
| | G3 | 114 ± 6 | 252 ± 39 | 435 ± 80 | 957 ± 174 | 6/6 | 64.5% | 0.450 | 0.001 |
| | G4 | 113 ± 6 | 254 ± 44 | 659 ± 114 | 1366 ± 199 | 6/6 | 47.2% | 0.385 | 0.011 |
| | G5 | 114 ± 5 | 233 ± 28 | 685 ± 84 | 1558 ± 187 | 6/6 | 39.1% | 0.436 | 0.024 |
| | G6 | 114 ± 6 | 277 ± 32 | 720 ± 137 | 1675 ± 301 | 6/6 | 34.2% | 0.332 | 0.092 |

B-TNFR2 Mice With MC38, 0.3 mg/kg In Vivo Efficacy

In another similar experiment, the dose of the anti-hTNFR2 antibody to be tested was reduced to 0.3 mg/kg. The mice (with MC38 tumor) were randomly assigned different groups when the tumors in the mice reached a volume of 100±50 mm³ (six mice in each group).

The mice were then injected with PBS as a control (G1), anti-hTNFR2 antibodies BC-1C3-lgG1 (G2), anti-mPD-1 (G3), anti-mCTLA4 (G4). The antibodies were given on the first day and the fourth day of each week by intraperitoneal injection at 0.3 mg/kg for 3 weeks (6 injections in total).

The weight of the mice was monitored throughout the experiment. The weight of mice in different groups all increased and there was no significant difference among the groups (P>0.05). The average weight of each group at the time of group assignment (D0) was in the range of 19.5 g-19.8 g. At the end of the experiment (25 days after group assignment, D25), the average weight was in the range of 21.6 g-23.3 g, and the weight change was in the range of 108.9%-118.2%. Similar to the previous results, the results showed that the anti-TNFR2 antibodies were well tolerated and not toxic to the mice.

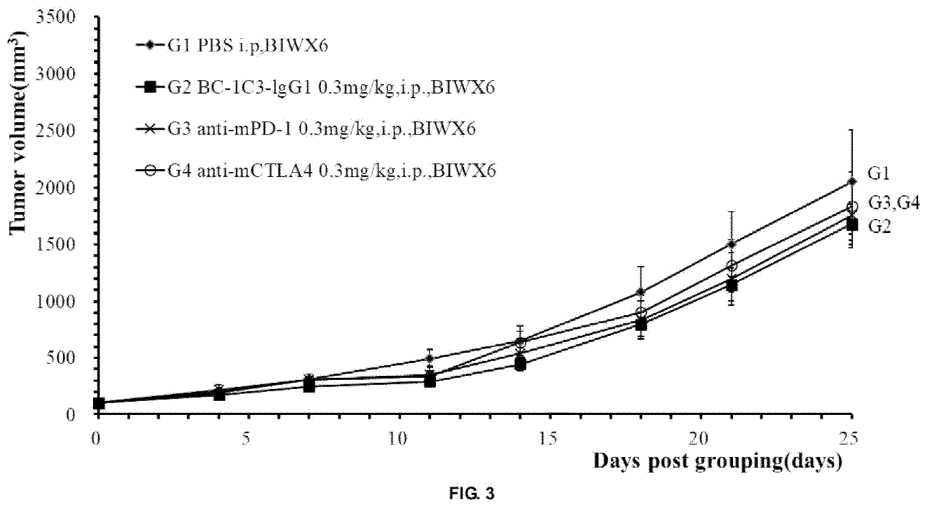

The tumor size (FIG. 3) showed significant difference in groups treated with the anti-hTNFR2 antibodies. The TGI_{TV}% at D25 (25 days after group assignment) for each treatment group was calculated as shown in Table 10 below. BC-1C3-lgG1 (G2) showed the best efficacy at this dose, and the efficacy was better than the positive control (G3, G4).

TABLE 10

| | | | | | | | | P value | |
|---|---|---|---|---|---|---|---|---|---|
| | | Tumor volume(mm³) | | | | | TGI_{TV} | Body | Tumor |
| | | D 0 | D 7 | D 18 | D 25 | Survival | % | weight | Volume |
| Control | G1 | 101 ± 5 | 311 ± 23 | 1083 ± 224 | 2053 ± 457 | 5/6 | n.a. | n.a. | n.a. |
| Treat | G2 | 101 ± 6 | 245 ± 33 | 796 ± 108 | 1680 ± 177 | 6/6 | 19.1% | 0.431 | 0.465 |
| | G3 | 102 ± 6 | 309 ± 48 | 835 ± 167 | 1758 ± 289 | 5/6 | 15.1% | 0.489 | 0.598 |
| | G4 | 101 ± 4 | 304 ± 32 | 904 ± 146 | 1838 ± 300 | 5/6 | 11.0% | 0.406 | 0.716 |

B-TNFR2 Mice With B16F10, 10 mg/kg In Vivo Efficacy

B16F10 tumor cells (melanoma cell) were injected subcutaneously into B-TNFR2 mice. When the tumors in the mice reached a volume of 100±50 mm³, the mice were randomly assigned to different groups based on the volume of the tumor (six mice in each group).

The mice were then injected with PBS as a control (G1), anti-hTNFR2 antibodies BC-1A8-IgG1 (G2), BC-1C3-lgG1 (G3), BC-1F10-lgG1 (G4), BC-1F4-lgG1 (G5), BC-1B6-lgG1 (G6). The antibodies were given on the first day and the fourth day of each week by intraperitoneal injection at 10 mg/kg for 2 weeks (3 injections in total).

The weight of the mice was monitored throughout the experiment. The weight of mice in different groups all increased and there was no difference among the groups (P>0.05). At the time of group assignment (D0), the average weight of each group was in the range of 19.2 g-19.7 g. On the 10th day (D10) after group assignment, the average weight of each group was the range of 22.1 g-24.5 g, and the weight change was the range of 114.3%-126.8%. The results showed that the anti-hTNFR2 antibodies were well tolerated and not toxic to the mice.

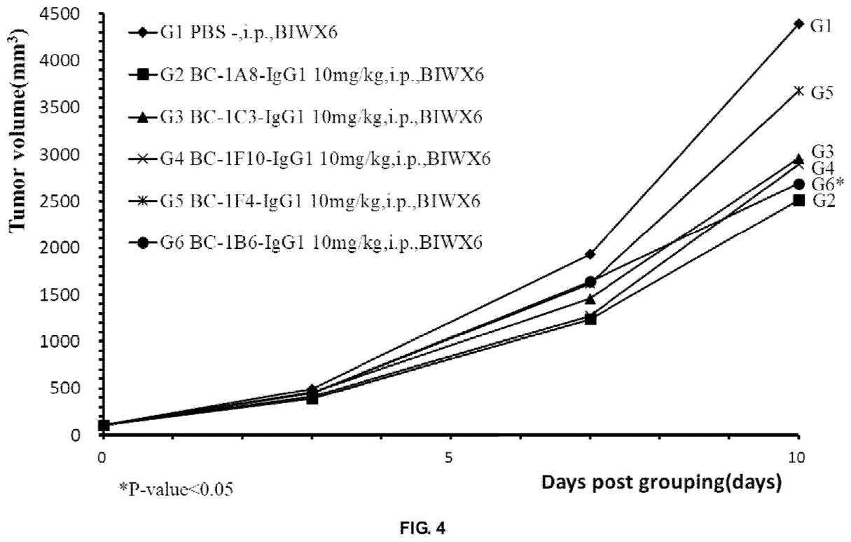

The tumor size showed significant difference in groups treated with the anti-TNFR2 antibodies (FIG. 4). BC-1A8-IgG1 (G2), BC-1C3-lgG1 (G3), BC-1F10-lgG1 (G4), and BC-1B6-lgG1 (G6) all showed anti-tumor efficacy in the B16F10 melanoma model. The TGI_{TV}% at Day 10 (10 days post grouping) for each treatment group is shown in Table 11 below.

TABLE 11

| | | | | | | | | P value | |
|---|---|---|---|---|---|---|---|---|---|
| | | Tumor volume(mm³) | | | | | TGI_{TV} | Body | Tumor |
| | | D 0 | D 3 | D 7 | D 10 | Survival | % | weight | Volume |
| Control | G1 | 105 ± 5 | 492 ± 66 | 1928 ± 290 | 4386 ± 500 | 5/6 | n.a. | n.a. | n.a. |
| Treat | G2 | 104 ± 5 | 391 ± 36 | 1240 ± 316 | 2515 ± 833 | 6/6 | 43.7% | 0.292 | 0.101 |
| | G3 | 105 ± 5 | 459 ± 61 | 1458 ± 245 | 2957 ± 401 | 6/6 | 33.4% | 0.698 | 0.050 |
| | G4 | 105 ± 4 | 413 ± 61 | 1275 ± 318 | 2895 ± 752 | 6/6 | 34.8% | 0.437 | 0.150 |
| | G5 | 105 ± 4 | 446 ± 20 | 1615 ± 2235 | 3674 ± 546 | 6/6 | 16.6% | 0.608 | 0.364 |
| | G6 | 105 ± 3 | 452 ± 41 | 1640 ± 548 | 2688 ± 518 | 5/6 | 39.7% | 0.436 | 0.046 |

B-TNFR2 Mice With MC38, 25 mg/kg In Vivo Efficacy and Toxicity

In another similar experiment, the dose of the anti-hTNFR2 antibody to be tested was adjusted to 25 mg/kg to test in vivo efficacy and toxicity. MC38 cancer tumor cells (colon adenocarcinoma cell) were injected subcutaneously in B-TNFR2 mice. When the tumors in the mice reached a volume of 100±50 mm³, the mice were randomly assigned to different groups based on the volume of the tumor (four mice in each group).

The mice were then injected with PBS as a control (G1), anti-hTNFR2 antibodies BC-1A8-IgG1 (G2), BC-1C3-IgG1 (G3), BC-1F10-IgG1 (G4), BC-1F4-IgG1 (G5), BC-1B6-IgG1 (G6). The antibodies were given on the day of group assignment (D0) and day 3 after group assignment (D3) by intraperitoneal injection at 25 mg/kg (2 injections in total).

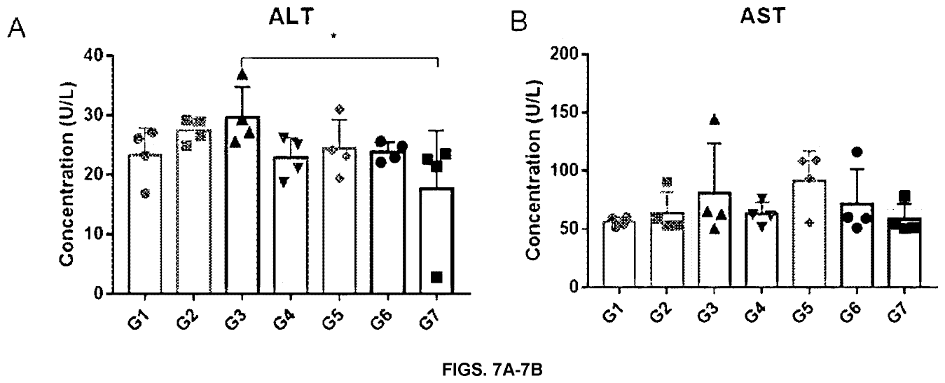

The weight of the mice was monitored throughout the experiment. The weight of mice in different groups all increased and there was no significant difference among the groups. The average weight of each group was in the range of 18.5 g-19.2 g at the time of group assignment. At the end of the experiment (21 days after group assignment), the average weight of each group was in the range of 21.1 g-23.3 g, and the weight change was between 110.0%-122.7%. On the 5th day (D5) after group assignment, the peripheral blood of mice was taken to test the blood biochemical indicators (AST, ALT). The results of biochemical index test on D5 (FIG. 7A-7B) showed that, compared with the control, ALT and AST did not change significantly. Similar to the previous results, the results showed that the 25 mg/kg of anti-TNFR2 antibodies were well tolerated and not toxic to the mice.

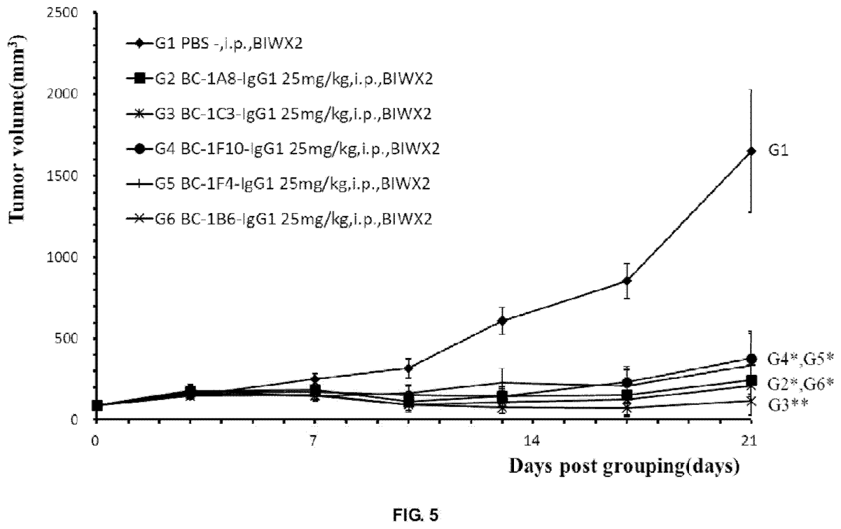

The tumor size (FIG. 5) showed significant difference in groups treated with the anti-TNFR2 antibodies. Compared with the control group G1, tumor volume was significantly reduced in the treatment groups treated with anti-hTNFR2 antibodies BC-1A8-IgG1 (G2), BC-1C3-IgG1 (G3), BC-1F10-IgG1 (G4), BC-1F4-IgG1 (G5) and BC-1B6-IgG1 (G6). TGI_{TV}% was between 81.6%-98.4%, showing obvious tumor suppression effect. The TGI_{TV}% on Day 21 (21 days after group assignment) for each treatment group was also calculated as shown in Table 12 below.

TABLE 12

| | | | | | | | | | P value | |
|---|---|---|---|---|---|---|---|---|---|---|
| | | Tumor volume(mm³) | | | | | Tumor | TGI_{TV} | Body | Tumor |
| | | Day 0 | Day 7 | Day 13 | Day 21 | Survival | free | % | weight | Volume |
| Control | G1 | 92 ± 7 | 251 ± 34 | 609 ± 83 | 1650 ± 376 | 4/4 | 0 | n.a. | n.a. | n.a. |
| Treat | G2 | 91 ± 10 | 186 ± 39 | 147 ± 45 | 246 ± 86 | 4/4 | 0 | 90.1% | 0.106 | 0.011 |
| | G3 | 91 ± 6 | 149 ± 31 | 77 ± 42 | 117 ± 93 | 4/4 | 1 | 98.4% | 0.036 | 0.007 |
| | G4 | 91 ± 7 | 173 ± 31 | 144 ± 58 | 378 ± 171 | 4/4 | 1 | 81.6% | 0.060 | 0.022 |
| | G5 | 92 ± 11 | 145 ± 28 | 228 ± 89 | 337 ± 194 | 4/4 | 1 | 84.3% | 0.039 | 0.021 |
| | G6 | 91 ± 10 | 152 ± 23 | 110 ± 71 | 210 ± 180 | 4/4 | 1 | 92.4% | 0.144 | 0.014 |

hTNFα/hTNFR2 Mice With MC38, 3 mg/kg In Vivo Efficacy and Toxicity

The humanized TNFα mouse model was engineered to express a human TNFα protein (SEQ ID NO: 79) wherein the coding sequence of the mouse TNFR2 protein was replaced with the corresponding human coding sequence. A double humanized TNFα/TNFR2 mouse model (B-hTNFα/hTNFR2 mice) was also generated by crossing the TNFα humanized mice with TNFR2 humanized mice. A detailed description regarding humanized TNFα mouse models can be found in PCT/CN2020/072714; which is incorporated herein by reference in its entirety.

Similar to the previous in vivo drug efficacy experiments, these anti-hTNFR2 antibodies were tested for their effects on tumor growth in vivo in a double humanized TNFα/TNFR2 mice model. In each group, double humanized TNFα/TNFR2 mice were injected with phosphate-buffered saline (PBS, G1), BC-1C3-lgG1 (G2) and anti-mPD-1 (G3) by intraperitoneal (i.p.) administration. The antibodies were given twice a week by intraperitoneal injection at 3 mg/kg for 3 weeks (6 injections in total).

The weight and the tumor size were monitored throughout the experiment. The weight of mice in different groups all increased and there was no significant difference among the groups (P>0.05). The average weight of each group at the time of group assignment (D0) was in the range of 20.0 g-20.6 g. At the end of the experiment (D28), the average weight was in the range of 23.5 g-25.1 g, and the weight change was in the range of 115.7%-127.2%. Similar to the previous results, the results showed that the anti-hTNFR2 antibodies were well tolerated and not toxic to the mice.

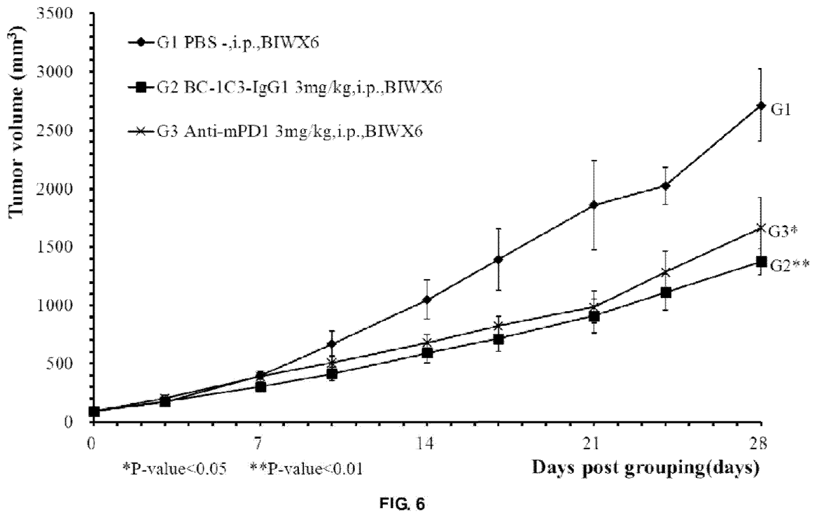

The tumor size (FIG. 6) showed significant difference in groups treated with the anti-hTNFR2 antibody. The TGI_{TV}% at D28 for each treatment group was calculated as shown in Table 13 below. BC-1C3-lgG1 (G2) showed the best efficacy at this dose, and the efficacy was better than the positive control (G3).

TABLE 13

| | | | | | | | | P value | |
|---|---|---|---|---|---|---|---|---|---|
| | | Tumor volume(mm³) | | | | | TGI_{TV} | Body | Tumor |
| | | D 0 | D 10 | D 21 | D 28 | Survival | % | weight | Volume |
| Control | G1 | 91 ± 5 | 669 ± 108 | 1861 ± 384 | 2713 ± 309 | 5/6 | n.a. | n.a. | n.a. |
| Treat | G2 | 91 ± 7 | 411 ± 56 | 910 ± 147 | 1375 ± 113 | 6/6 | 51.0% | 0.333 | 0.003 |
| | G3 | 91 ± 6 | 508 ± 64 | 987 ± 140 | 1664 ± 261 | 6/6 | 40.0% | 0.250 | 0.035 |

B-TNFR2 Mice With MC38, 3 mg/kg In Vivo Efficacy

Similar to the aforementioned in vivo drug efficacy experiments, MC38 cancer tumor cells were injected subcutaneously in B-TNFR2 mice. When the tumors in the mice reached a volume of 100 mm³, the mice were randomly assigned to different groups based on the volume of the tumor (six mice in each group). The mice were injected with PBS as a control (G1), 3 mg/kg BC-1C3-lgG1 (G2), 3 mg/kg anti-mPD-1 (G3), combination of 3 mg/kg BC-1C3-lgG1 and 3 mg/kg anti-mPD-1 (G4), 3 mg/kg atezolizumab analog (G5), or combination of 3 mg/kg BC-1C3-lgG1 and 3 mg/kg atezolizumab analog (G6). The frequency of administration was twice a week (6 administrations in total).

Atezolizumab is a humanized anti-PD-L1 monoclonal antibody developed by Genentech (VH SEQ ID NO: 80; VL SEQ ID NO: 81).

The weight and the tumor size were monitored throughout the experiment. The weight of mice in different groups all increased and there was no significant difference among the groups (P>0.05). The average weight of each group at the time of group assignment (D0) was in the range of 18.9 g-19.3 g. At the end of the experiment (D24), the average weight was in the range of 22.4-24.2 g, and the weight change was in the range of 118.6%-125.3%. Similar to the previous results, the results showed that the anti-hTNFR2 antibodies were well tolerated and not toxic to the mice.

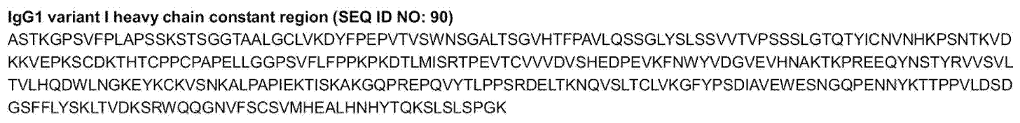

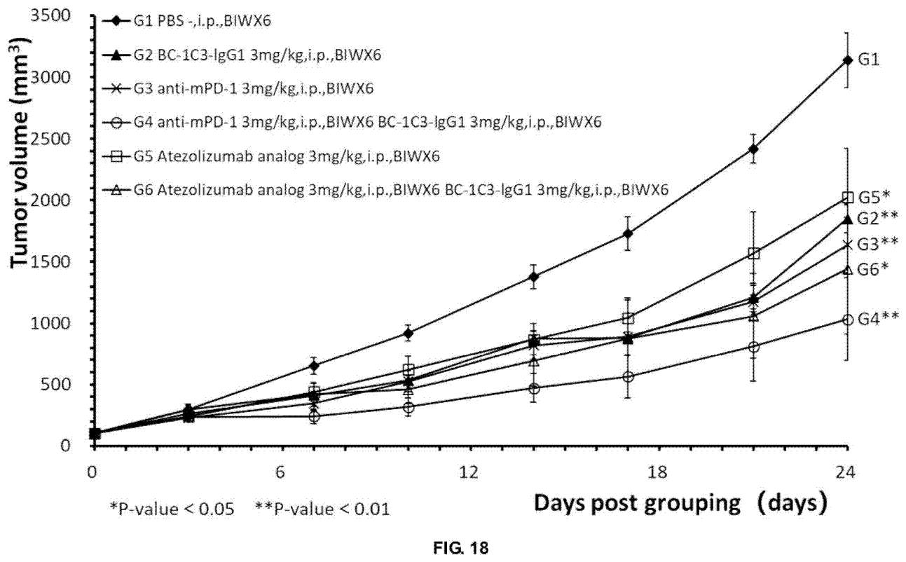

The tumor size in groups treated with the antibodies is shown in FIG. 18. The TGI_{TV}% at D24 for each treatment group was calculated as shown in Table 14 below. Compared with the control group, the tumor growth in the treatment groups was inhibited to different extents. Among the treatment groups, combination of anti-PD-1 antibody and anti-hTNFR2 antibody (G4) inhibited tumor growth with superior efficacy. Similarly, combination of anti-PD-L1 antibody atezolizumab analog and anti-hTNFR2 antibody (G6) also exhibited better tumor growth inhibition than anti-hTNFR2 antibody (G2) and anti-PD-L1 antibody atezolizumab analog (G5).

TABLE 14

| | | | | | | | | P value | |
|---|---|---|---|---|---|---|---|---|---|
| | | Tumor volume(mm³) | | | | | TGI_{TV} | Body | Tumor |
| | | D 0 | D 7 | D 14 | D 24 | Survival | % | weight | Volume |
| Control | G1 | 104 ± 6 | 652 ± 67 | 1378 ± 95 | 3137 ± 221 | 6/6 | n.a. | n.a. | n.a. |
| Treat | G2 | 104 ± 8 | 411 ± 30 | 875 ± 58 | 1849 ± 112 | 6/6 | 42.5% | 0.333 | 4.05E−04 |
| | G3 | 104 ± 7 | 348 ± 58 | 819 ± 119 | 1638 ± 224 | 6/6 | 49.4% | 0.312 | 7.72E−04 |
| | G4 | 104 ± 7 | 241 ± 59 | 472 ± 117 | 1034 ± 336 | 6/6 | 69.3% | 0.426 | 3.88E−04 |
| | G5 | 104 ± 8 | 440 ± 70 | 869 ± 128 | 2022 ± 401 | 6/6 | 36.8% | 0.368 | 0.035 |
| | G6 | 104 ± 7 | 419 ± 95 | 693 ± 206 | 1441 ± 530 | 6/6 | 55.9% | 0.986 | 0.015 |

B-TNFR2 Mice With GL261, 1-10 mg/kg In Vivo Efficacy

GL261 tumor cells (glioblastoma cell) were injected subcutaneously in B-TNFR2 mice. When the tumors in the mice reached a volume of about 80 mm³, the mice were randomly placed into different groups based on the volume of the tumor (six mice in each group).

The mice were then injected with PBS as a control (G1), 1 mg/kg BC-1C3-IgG1 (G2), 3 mg/kg BC-1C3-IgG1 (G3), 10 mg/kg BC-1C3-IgG1 (G4), or 1 mg/kg anti-mPD-1 (G5). The antibodies were given on the first day and the fourth day of each week by intraperitoneal injection for 3 weeks (6 injections in total).

The weight of the mice were monitored during the entire treatment period. The weight of mice in different groups all increased, with no statistically significant differences (P>0.05). On the day of group assignment (Day 0; “D0”), the average weight of each group was in the range of 19.9 g-20.6 g. At the end of the experiment (24 days after group assignment, D24) the average weight of each group was in the range of 22.5 g-24.2 g, the weight changes being in the range of 109.4%-118.3%. The results showed that the anti-hTNFR2 antibodies were well tolerated and not toxic to the mice.

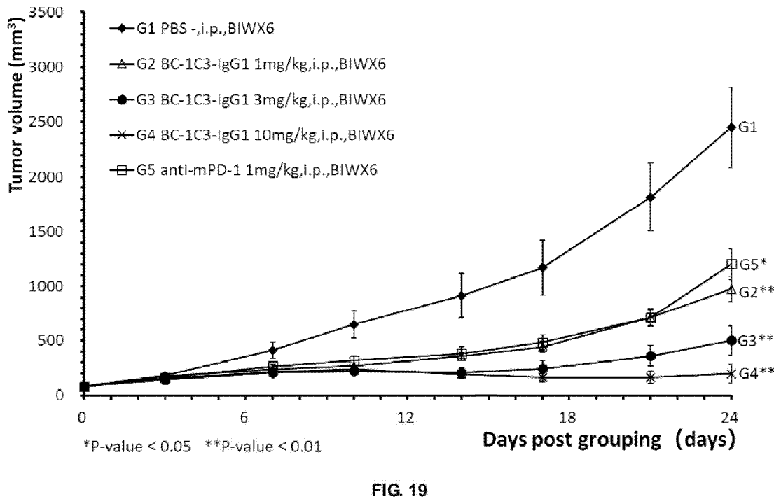

The tumor size data in groups treated with the antibodies is shown in FIG. 19. The TGI_{TV}% at D24 for each treatment group was calculated as shown in Table 15 below. BC-1C3-IgG showed better anti-tumor effects than anti-mPD-1 antibody at 1 mg/kg dosage. In addition, BC-1C3-IgG1 exhibited a dose-dependent anti-tumor effect (the higher the dose level, the better anti-tumor effect).

TABLE 15

| | | | | | | | | P value | |
|---|---|---|---|---|---|---|---|---|---|
| | | Tumor volume(mm³) | | | | | TGI_{TV} | Body | Tumor |
| | | D 0 | D 7 | D 14 | D 24 | Survival | % | weight | Volume |
| Control | G1 | 82 ± 4 | 413 ± 73 | 916 ± 204 | 2451 ± 369 | 6/6 | n.a. | n.a. | n.a. |
| Treat | G2 | 82 ± 5 | 235 ± 11 | 356 ± 31 | 976 ± 116 | 6/6 | 62.3% | 0.203 | 3.38E−03 |
| | G3 | 82 ± 4 | 210 ± 13 | 209 ± 42 | 502 ± 135 | 6/6 | 82.3% | 0.021 | 5.67E−04 |
| | G4 | 82 ± 5 | 217 ± 17 | 194 ± 35 | 199 ± 84 | 6/6 | 95.1% | 0.035 | 1.40E−04 |
| | G5 | 82 ± 5 | 267 ± 20 | 382 ± 60 | 1204 ± 139 | 6/6 | 52.6% | 0.429 | 0.010 |

B-TNFR2 Mice With MC38, 3 mg/kg In Vivo Efficacy

BI-1808 (VH SEQ ID NO: 82; VL SEQ ID NO: 83) is a monoclonal antibody targeting tumor necrosis factor receptor superfamily member 1B (TNFR2) developed by BioInvent. The product is in early clinical development for the treatment of solid tumors and cutaneous T-cell lymphoma (CTCL) as a single agent and in combination with pembrolizumab.

h600-25-108 (VH SEQ ID NO: 84; VL SEQ ID NO: 85) is a humanized anti-TNFR2 monoclonal antibody developed by Apexigen.

HFB3-1hz6-hG1 (VH SEQ ID NO: 86; VL SEQ ID NO: 87) is a monoclonal anti-TNFR2 agonist antibody in phase I clinical trials at HiFiBIO Therapeutics for the treatment of advanced solid tumors.

Similar to the aforementioned in vivo drug efficacy experiments, MC38 tumor cells were injected subcutaneously in B-TNFR2 mice. When the tumors reached a volume of 100 mm³, the mice were randomly placed into different groups based on the volume of the tumor (six mice in each group). The mice were injected with PBS (G1), 3 mg/kg BC-1C3-lgG1 (G2), 3 mg/kg BI-1808 analog (G3), 3 mg/kg h600-25-108 analog (G4), or 3 mg/kg HFB3-1hz6-hG1 analog (G5). The frequency of administration was twice a week (6 administrations in total).

The weight and the tumor size were monitored throughout the experiment. The weight of mice in different groups all increased and there was no significant difference among the groups (P>0.05). The average weight of each group at the time of group assignment (D0) was in the range of 20.1 g-20.4 g. At the end of the experiment (D28), the average weight was in the range of 22.9-24.9 g, and the weight change was in the range of 113.2%-122.4%. Similar to the previous results, the results showed that the anti-hTNFR2 antibodies were well tolerated and not toxic to the mice.

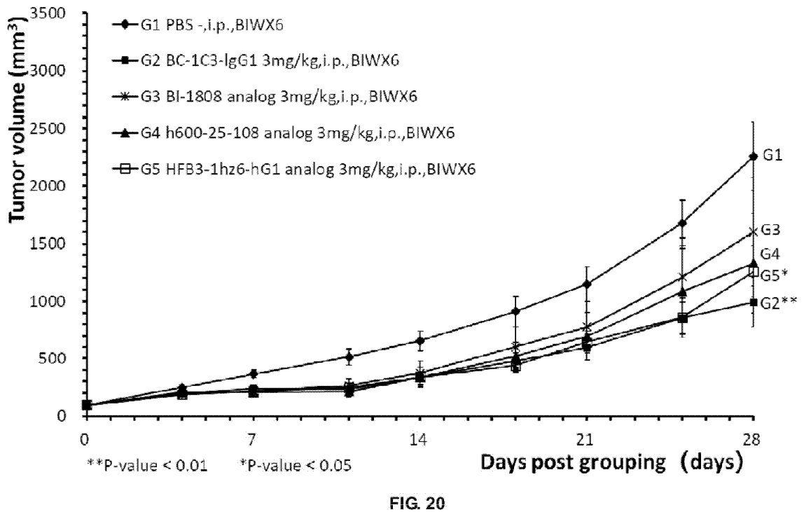

The tumor sizes are shown in FIG. 20. The TGI_{TV}% at D28 for each treatment group was calculated as shown in Table 16 below. The results showed that the BC-1C3-lgG1 antibody (G2) showed significantly better in vivo efficacy than BI-1808 analog (G3), h600-25-108 analog (G4), or HFB3-1hz6-hG1 analog (G5) at the same dose level.

TABLE 16

| | | | | | | | P value | |
|---|---|---|---|---|---|---|---|---|
| | | Tumor volume(mm³) | | | | TGI_{TV} | Body | Tumor |
| | | D 0 | D 14 | D 28 | Survival | % | weight | Volume |
| Control | G1 | 95 ± 3 | 654 ± 85 | 2257 ± 297 | 6/6 | n.a. | n.a. | n.a. |
| Treat | G2 | 95 ± 4 | 335 ± 27 | 992 ± 213 | 6/6 | 58.5% | 0.082 | 6.07E−03 |
| | G3 | 95 ± 4 | 375 ± 99 | 1602 ± 470 | 6/6 | 30.3% | 0.468 | 0.266 |
| | G4 | 95 ± 4 | 340 ± 85 | 1330 ± 432 | 4/6 | 42.9% | 0.192 | 0.103 |
| | G5 | 95 ± 4 | 339 ± 43 | 1260 ± 221 | 6/6 | 46.1% | 0.057 | 0.022 |

B-hTNFa/hTNFR2 Mice With MC38, 3 mg/kg In Vivo Efficacy

h600-25-71 (VH SEQ ID NO: 88; VL SEQ ID NO: 89) is a humanized anti-TNFR2 monoclonal antibody developed by Apexigen.

Similar to the aforementioned in vivo drug efficacy experiments, MC38 tumor cells were injected subcutaneously in B-hTNFα/hTNFR2 mice. When the tumors reached a volume of 100 mm³, the mice were randomly placed into different groups based on the volume of the tumor (six mice in each group), and were injected with PBS (G1), 3 mg/kg BC-1C3-lgG1 (G2) or 3 mg/kg h600-25-71 analog (G3). The frequency of administration was twice a week (6 administrations in total).

The weight and the tumor size were monitored throughout the experiment. The weight of mice in different groups all increased and there was no significant difference among the groups (P>0.05). The average weight of each group at the time of group assignment (D0) was in the range of 21.0 g-21.1 g. At the end of the experiment (D24), the average weight was in the range of 23.8-24.8 g, and the weight change was in the range of 113.3%-117.5%. Similar to the previous results, the results showed that the anti-hTNFR2 antibodies were well tolerated and not toxic to the mice.

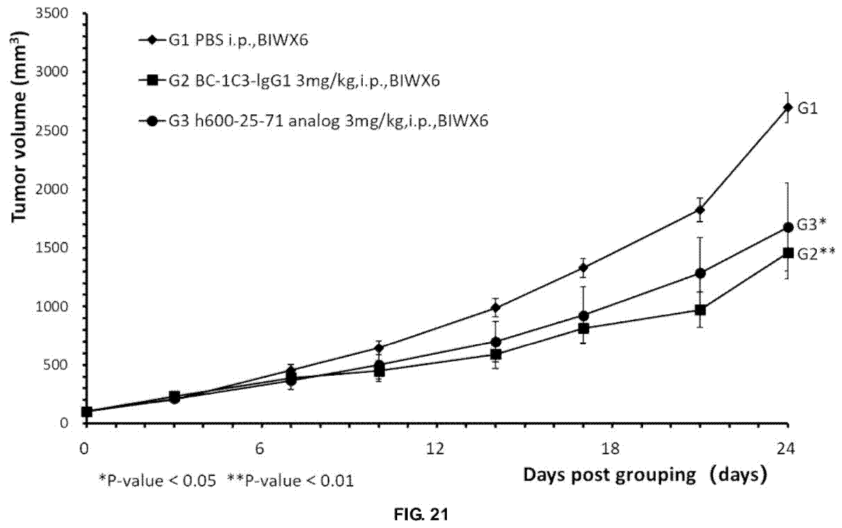

The tumor sizes are shown in FIG. 21. The TGI_{TV}% at D24 for each treatment group values are shown in Table 17 below. The results showed that BC-1C3-lgG1 exhibited significantly better in vivo efficacy than h600-25-71 analog.

TABLE 17

| | | | | | | | P value | |
|---|---|---|---|---|---|---|---|---|
| | | Tumor volume(mm³) | | | | TGI_{TV} | Body | Tumor |
| | | D 0 | D 10 | D 24 | Survival | % | weight | Volume |
| Control | G1 | 102 ± 2 | 645 ± 59 | 2696 ± 125 | 6/6 | n.a. | n.a. | n.a. |
| Treat | G2 | 102 ± 3 | 449 ± 89 | 1461 ± 222 | 6/6 | 47.6% | 0.333 | 0.001 |
| | G3 | 102 ± 3 | 500 ± 121 | 1678 ± 376 | 6/6 | 39.3% | 0.461 | 0.028 |

### Example 4. PK and TILS Analysis

B-TNFR2 Mice With MC38 Model, 10 mg/kg, PK Analysis

The pharmacokinetic clearance rate of anti-TNFR2 antibody was determined in humanized TNFR2 mice. MC38 cells (5× 105) were injected subcutaneously into humanized TNFR2 mice, and the mice were divided into 8 groups (n=4) when the tumor grew to 300mm³. 10 mg/kg of BC-1C3-IgG1 (G2), BC-1F4-IgG1 (G3), BC-1B6-IgG1 (G4), BC-1C3-IgG1-SI (G5), BC-1F4-IgG1-SI (G6), BC-1B6-IgG1-SI (G7), or isotype control IgG1 (G1) was administered by intravenous injection. Blood samples were collected 15 min, 6 hr, 24 hr, day 3, day 5, and day 7 after administration.

The serum levels of human antibodies were determined by sandwich ELISA enzyme-linked immunoassay. In short, goat polyclonal anti-human IgG (Fc specific) capture antibody (Jackson ImmunoResearch, Cat #: 109-036-098) was diluted to a final concentration of 2.0 μg/mL with 0.1 M carbonate buffer (pH 9.6), added to a 96-well plate (ELISA plate) at 100 μL/well, and incubated overnight at 4° C. Then, 200 μL of blocking buffer (2% BSA) was added to each well. The wells were sealed and incubated at room temperature for 1 hr. After washing the plate with a plate washer, horseradish peroxidase (HRP)-conjugated goat polyclonal anti-human IgG (Fc specific) antibody (Jackson ImmunoResearch, Cat #: 109-005-088), was added at 100 μL/well to each well of the ELISA plate, and incubated at 37° C. for 1 hr. After washing the plate, tetramethylbenzidine (TMB) solution was added at 100 μL/well to the 96-well plate as the substrate to react with HRP. After incubating at room temperature in the dark, 100 μL stop solution (Beyotime, Cat #: P0215) was added to each well. A microplate reader was used to read the absorbance value of each well at wavelengths of 450 nm and 630 nm. The data analysis software Gen5 was used to analyze the data. The absorbance value and corresponding concentration of the calibration sample prepared by each test product was used to create a standard curve with four parameters. And the standard curve was used to calculate the antibody concentration of each serum sample. A drug concentration-time curve was created using the calculated sample concentration at each time point. Phoenix winnolin 8.3 was used to calculate the pharmacokinetic parameters. The results are shown in the following Table 18.

TABLE 18

| | | T_{1/2} | Cₘₐₓ | AUC_{0-7 day} | CL |
|---|---|---|---|---|---|
| Group | | (day) | (ug/mL) | (hr*ug/mL) | (ml/hr/kg) |
| G1 | MEAN | 11.05 | 185.95 | 418.90 | 9.13 |
| | CV | 18.9% | 5.2% | 8.8% | 15.3% |
| G2 | MEAN | 2.99 | 152.90 | 250.93 | 28.85 |
| | CV | 26.3% | 5.4% | 5.4% | 15.3% |
| G3 | MEAN | 2.38 | 134.41 | 168.89 | 47.07 |
| | CV | 12.3% | 9.4% | 3.0% | 6.9% |
| G4 | MEAN | 1.89 | 134.99 | 144.27 | 61.19 |
| | CV | 15.3% | 6.0% | 13.5% | 14.5% |
| G5 | MEAN | 2.00 | 130.01 | 152.27 | 56.43 |
| | CV | 3.2% | 4.0% | 7.8% | 7.9% |
| G6 | MEAN | 1.69 | 130.35 | 156.65 | 58.09 |
| | CV | 17.8% | 8.4% | 15.5% | 16.9% |
| G7 | MEAN | 0.86 | 111.90 | 77.90 | 119.02 |
| | CV | 92.0% | 4.3% | 12.2% | 8.7% |

T_{1/2}: Terminal Half Life
Cₘₐₓ: Max Concentration
AUC_{0-7 day}: Area under Blood Concentration-time Curve0-7 day
CL: Clearance

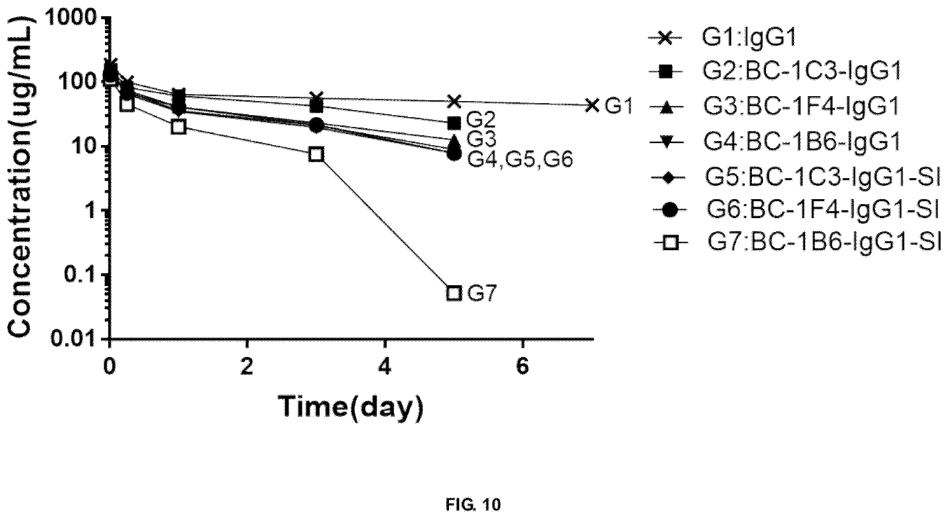

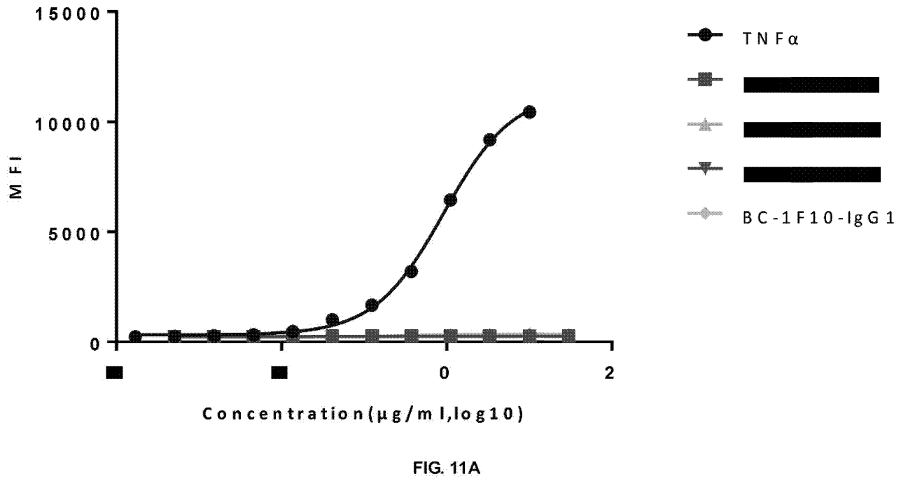

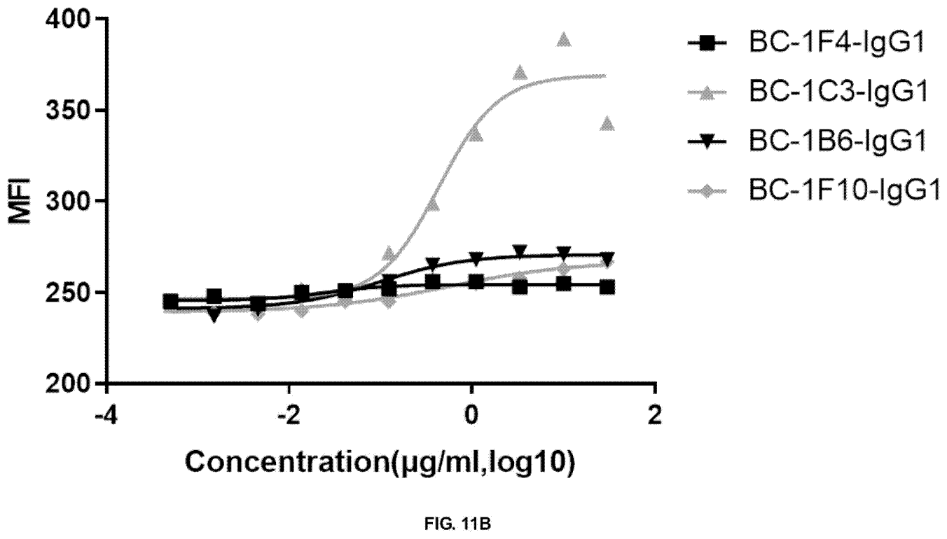

The above results show that after injection of different antibodies, the concentration of antibodies in the serum of the TNFR2 humanized mice decreased with time (see FIG. 10), which is consistent with pharmacokinetic characteristics. The longest half-life T1/2 of BC-1C3-IgG1 (G2) antibody in mice was 2.99 days. The shortest half-life of BC-1B6-IgG1-SI (G7) antibody in mice was 0.86 days. The half-life of other antibodies in mice was within the range of 1.69 days to 2.38 days, which was relatively close. By the end of the seventh day of sampling, the area under curve (AUC) of the drug concentration-time curve of the BC-1C3-IgG1 (G2) antibody was 250.93 hr*μg/mL, which was larger than the AUC of other antibodies (AUCO-7day 77.90 ˜168.89 hr*μg/mL). The clearance rate (CL) of BC-1C3-IgG1 (G2) antibody was 28.85 ml/hr/kg, and the CL of other antibodies was in the range of 47.07-61.19 ml/hr/kg. The results showed that compared with other antibodies, BC-1C3-IgG1 (G2) has a lower clearance efficiency, and the metabolism of BC-1C3-IgG1 (G2) antibody in mice was slower.

### Example 5. Reporter Cell Activation and Binding Assay

### The Activation Effect on the Reporter Cells

The experiment was performed to test whether the anti-TNFR2 antibodies can active the TNFR2 pathway.

Human TNFα protein (Sino Biological Inc., Cat #: 10602-HNAE) were serially diluted (3-fold) with a highest concentration of 10 ng/mL as the positive control. Anti-TNFR2 antibodies BC-1F4-IgG1, BC-1C3-IgG1, BC-1B6-IgG1 and BC-1F10-IgG1 were serially diluted (3-fold) with a highest concentration of 60 μg/mL. Jurkat-GFP-TNFR2 cells were seeded in a 96-well plate (cell density 1×10⁵ cells/well, 100 μL per well), then 100 μl Human TNFα protein or 100 μl anti-TNFR2 antibody were added to each well and incubated at 37° C. overnight. After the incubation, the plate was taken out, and transferred to a 96-well plate. Each well was washed with 150 μl PBS. The supernatant was discarded. 100 μL PBS was added to each well to re-suspend the cells. The plate was then placed in a luminescence detector to detect the fluorescence signal. If the antibody can active TNFR2, the reporter cells will report a GFP signal.

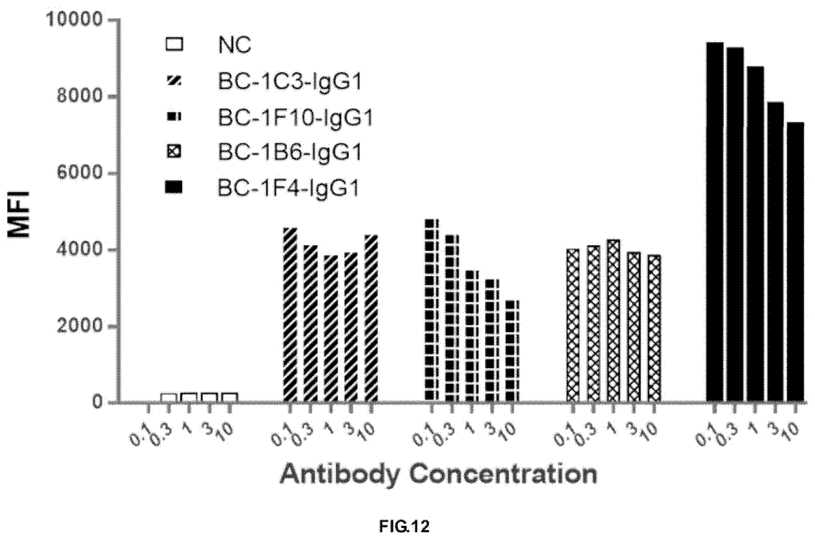

As shown in FIG. 11A, no fluorescence signal was detected when the anti-TNFR2 antibodies BC-1F4-IgG1, BC-1C3-IgG1, BC-1B6-IgG1 and BC-1F10-IgG1 were present. While BC-1C3-IgG1 exhibited weak reporter cell activation (FIG. 11B).

### The Blocking Effect on the Reporter Cells

The experiment was performed to test whether the anti-TNFR2 antibodies can block the binding between TNFR2 and its ligand TNFα.

Reporter Jurkat-GFP-TNFR2 cells were seeded in a 96-well plate (cell density 1×10⁵ cells/well). TNFα protein was diluted to 1 ng/mL. The anti-TNFR2 antibodies BC-1F4-IgG1, BC-1C3-IgG1, BC-1B6-IgG1 and BC-1F10-IgG1 were serially diluted (3-fold) with the highest concentration of 10 μg/mL. 50 μl Human TNFα protein and 50 μl antibody were added to each well and incubated at 37° C. for 24 hours. After the incubation, the plate was taken out, and transferred to a 96-well plate. Each well was washed with 150 μl PBS. The supernatant was discarded. 100 μL PBS was added to each well to re-suspend the cells. The signals for GFP were determined by flow cytometry.

As shown in FIG. 12, when the concentration of the anti-TNFR2 antibodies BC-1F10-IgG1 and BC-1F4-IgG1 increased, the GFP signal (indicating cells binding to TNFα) decreased (y axis), suggesting that the binding between human TNFα and TNFR2 was blocked by the anti-TNFR2 antibodies BC-1F10-IgG1 and BC-1F4-IgG1.

### Example 6. In Vivo Toxicity Experiment (Non-Tumor Bearing Model)

TNFR2 humanized mice (6-8 weeks) were randomly divided into control group and treatment group (4 mice in each group) according to their body weight. The control group was injected with an equal volume of PBS, and the administration group is injected with anti-h TNFR2 Antibody (BC-1C3-IgG1, BC-1F4-IgG1 or BC-1B6-IgG1,) or CTLA4 antibody (anti-mCTLA4). The injection dose of anti-hTNFR2 antibody and CTLA4 antibody is 30 mg/kg or 100 mg/kg. The frequency of administration is once a week for a total of 4 administrations. The specific dosage, mode and frequency of administration are shown in the Table 19 below. In the experiment, the change of body weight and any abnormality were monitored. On day 1, day 8, day 15, day 22, and day 28 after group assignment, blood biochemical indicators were monitored. The blood biochemical indicators include: aspartate aminotransferase (AST), alanine aminotransferase (ALT), alkaline phosphatase (ALP), creatine kinase (CK), albumin (ALB), total protein (TP), amylase (AMY), urea (UREA), creatinine (CREA), glucose (GLU), triglycerides (TG), total cholesterol (TC), High-density lipoprotein cholesterol (HDL-C), low-density lipoprotein cholesterol (LDL-C), calcium (Ca), inorganic phosphorus (P)). On day 28 after group assignment, blood routine test was conducted. The blood routine test includes testing on white blood cell count (WBC), red blood cell (RBC), hemoglobin (HGB), Hematocrit (HCT), average red blood cell volume (MCV), average red blood cell hemoglobin content (MCH), average red blood cell hemoglobin concentration (MCHC), PLT (platelet count), lymphocytes (LYMPH #), lymphocyte percentage (LYMPH %), Monocytes (MONO #), percentage of monocytes (MONO %), percentage of neutrophils (NEUT %). At the end of the experiment, the mouse heart, liver, spleen, lung, kidney, and intestine were put into formalin for HE staining, and the weight of the liver, spleen, and kidney were measured.

TABLE 19

| Group | Drug | Treatment |
|---|---|---|
| G1 | PBS | Intraperitoneal injection, once a week, 4 times in total |
| G2 | BC-1C3-IgG1 | 30 mg/kg, Intraperitoneal injection, once a week, 4 times in total |
| G3 | BC-1C3-IgG1 | 100 mg/kg, Intraperitoneal injection, once a week, 4 times in total |
| G4 | BC-1F4-IgG1 | 30 mg/kg, Intraperitoneal injection, once a week, 4 times in total |
| G5 | BC-1F4-IgG1 | 100 mg/kg, Intraperitoneal injection, once a week, 4 times in total |
| G6 | BC-1B6-IgG1 | 30 mg/kg, Intraperitoneal injection, once a week, 4 times in total |
| G7 | BC-1B6-IgG1 | 100 mg/kg, Intraperitoneal injection, once a week, 4 times in total |
| G8 | anti-mCTLA4 | 30 mg/kg, Intraperitoneal injection, once a week, 4 times in total |
| G9 | anti-mCTLA4 | 100 mg/kg, Intraperitoneal injection, once a week, 4 times in total |

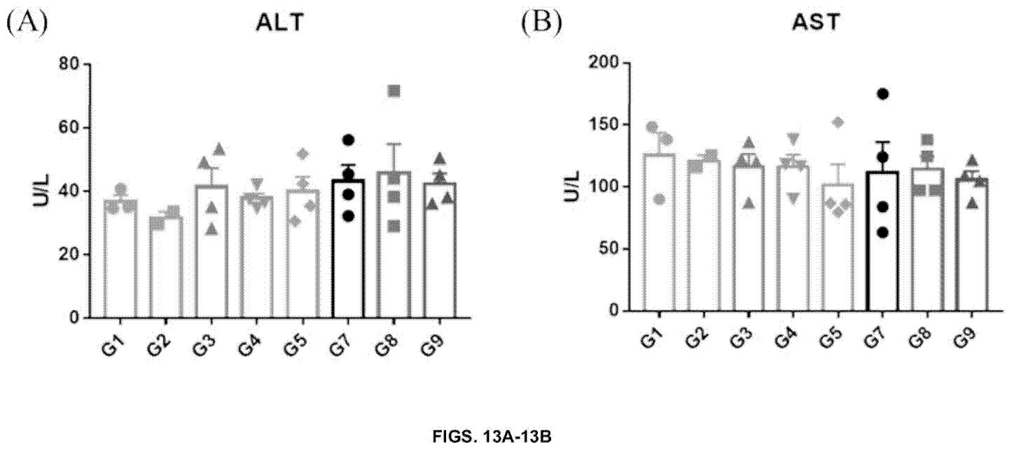

The results showed that the body weight of all mice in the control group and the treatment group showed an upward trend throughout the experimental period, and there was no significant difference in body weight changes among the groups. The blood biochemical indicator testing results (see FIGS. 13A-13B for exemplary blood biochemical indicators at day 28) and blood routine test results showed no significant difference compared with the control. Similar to the previous results, the in vivo toxicity results showed that the anti-hTNFR2 antibodies were well tolerated and not toxic to the mice.

### Example 7. TNFR2 Antibody Blocks the Inhibition of the Proliferation of CD8+ T Cells by Treg Cells

Human IgG1 (control) and anti-TNFR2 antibodies BC-1C3-IgG1 and HFB3-1hz6-hG1 analog were diluted to a final concentration of 20ng/mL. An anti-CD3 antibody (CD3, Acro Biosystems, Cat #: CDE-M120a) was used to coat a 96-well plate (10 μg/mL, 100 μl per well) overnight at 4° C. 100 μl PBMC cells (AllCells, Cat #: PB003F-C) labelled with CFSE (CellTrace™ M CFSE Cell Proliferation Kit, Thermo Fisher, Cat #: CDE-M120a) and 50 μl Treg cells (OriCell, Cat #: FPB009-4F-2) were added to each well. 40 μl BC-1C3-IgG1, 40 μl human IgG1 or 40 μl HFB3-1hz6-hG1 analog was added to each well and incubated at 37° C., 5% CO₂ for 120 hours. After 48h of incubation, 10 μl IL-2 (Acro Biosystems, Cat #: IL-2-H4113) was added to each well. After 120 hours of incubation, cells were collected and hCD8+ cells were determined by flow cytometry.

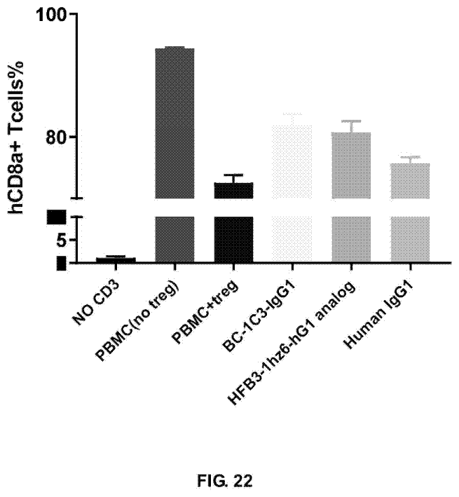

As shown in FIG. 22, BC-1C3-IgG1 blocked the inhibition of the proliferation of CD8+ T cells by Treg cells.

### Example 8. CD8+ T Cell Activation Assay

Human IgG1 (control), anti-TNFR2 antibodies BC-1C3-IgG1 and HFB3-1hz6-hG1 analog were serially diluted to 0.1 μg/mL, 1 μg/mL, and 10 μg/mL. An anti-CD3 antibody and the anti-TNFR2 antibodies were used to coat a 96-well plate overnight at 4° C. CD8+ T cells (OriCell) labelled with CFSE (CellTrace™ CFSE Cell Proliferation Kit, Thermo Fisher, Cat #: CDE-M120a) were added to each well (1×10⁵ cells/well, 100 μl per well), and hCD28 (BioXcell, Cat #: BE0248) was added to each well (1 μg/mL, 100 μl per well) and incubated at 37° C., 5% CO2 for 72 hours. The supernatant was collected to detect the secretion levels of human IL2 and human IFNγ, and the cells in the pellet were detected by flow cytometry.

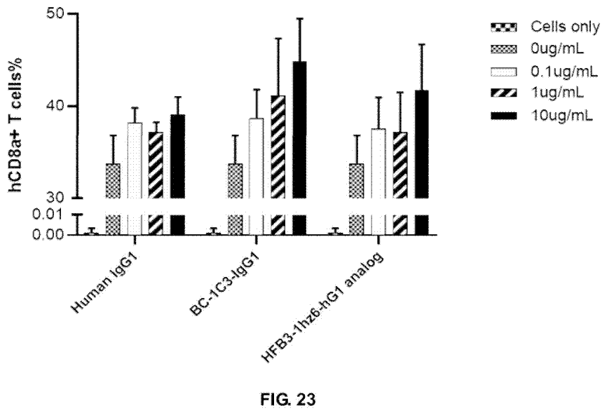

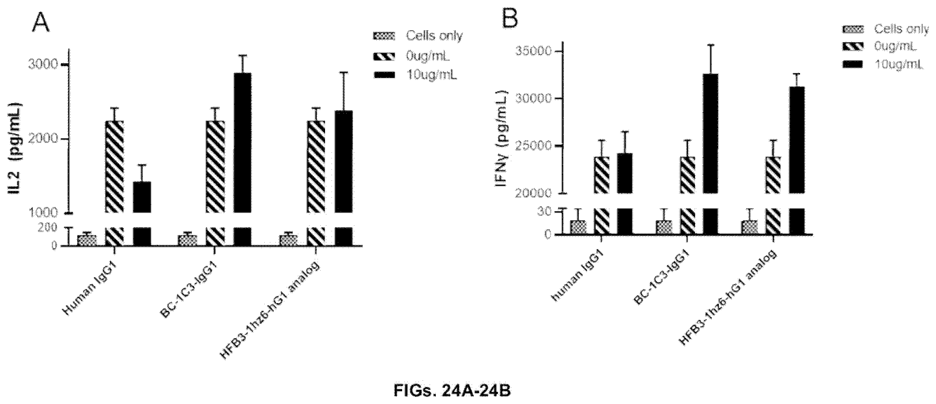

As shown in FIG. 23, CD8+ T cells increased with increasing concentrations of anti-TNFR2 antibody BC-1C3-IgG1. FIGS. 24A-24B show that human IL2 and IFNγ secretion increased after adding the anti-TNFR2 antibodies. The above results indicate that BC-1C3-IgG1 can promote the proliferation and activation of CD8+ T cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to TNFR2, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 6, 7, 8, respectively, and the selected VL CDRs 1, 2, 3 amino acid sequences are set forth in SEQ ID NOs: 9, 10, 11, respectively;
(2) the selected VH CDRs 1, 2, 3 amino acid sequences are set forth in SEQ ID NOs: 12, 13, 14, respectively, and the selected VL CDRs 1, 2, 3 amino acid sequences are set forth in SEQ ID NOs: 15, 16, 17, respectively;
(3) the selected VH CDRs 1, 2, 3 amino acid sequences are set forth in SEQ ID NOs: 18, 19, 20, respectively, and the selected VL CDRs 1, 2, 3 amino acid sequences are set forth in SEQ ID NOs: 21, 22, 23, respectively;
(4) the selected VH CDRs 1, 2, 3 amino acid sequences are set forth in SEQ ID NOs: 24, 25, 26, respectively, and the selected VL CDRs 1, 2, 3 amino acid sequences are set forth in SEQ ID NOs: 27, 28, 29, respectively;
(5) the selected VH CDRs 1, 2, 3 amino acid sequences are set forth in SEQ ID NOs: 30, 31, 32, respectively, and the selected VL CDRs 1, 2, 3 amino acid sequences are set forth in SEQ ID NOs: 33, 34, 35, respectively;
(6) the selected VH CDRs 1, 2, 3 amino acid sequences are set forth in SEQ ID NOs: 42, 43, 44, respectively, and the selected VL CDRs 1, 2, 3 amino acid sequences are set forth in SEQ ID NOs: 45, 46, 47, respectively;
(7) the selected VH CDRs 1, 2, 3 amino acid sequences are set forth in SEQ ID NOs: 48, 49, 50, respectively, and the selected VL CDRs 1, 2, 3 amino acid sequences are set forth in SEQ ID NOs: 51, 52, 53, respectively;
(8) the selected VH CDRs 1, 2, 3 amino acid sequences are set forth in SEQ ID NOs: 54, 55, 56, respectively, and the selected VL CDRs 1, 2, 3 amino acid sequences are set forth in SEQ ID NOs: 57, 58, 59, respectively;
(9) the selected VH CDRs 1, 2, 3 amino acid sequences are set forth in SEQ ID NOs: 60, 61, 62, respectively, and the selected VL CDRs 1, 2, 3 amino acid sequences are set forth in SEQ ID NOs: 63, 64, 65, respectively; or
(10) the selected VH CDRs 1, 2, 3 amino acid sequences are set forth in SEQ ID NOs: 66, 67, 68, respectively, and the selected VL CDRs 1, 2, 3 amino acid sequences are set forth in SEQ ID NOs: 69, 70, 71, respectively.

2. The antibody or antigen-binding fragment thereof of claim 1, wherein the VH comprises CDRs 1, 2, 3 with the amino acid sequences set forth in SEQ ID NOs: 6, 7, and 8 respectively, and the VL comprises CDRs 1, 2, 3 with the amino acid sequences set forth in SEQ ID NOs: 9, 10, and 11, respectively according to Kabat numbering.

3. The antibody or antigen-binding fragment thereof of claim 1, wherein the VH comprises CDRs 1, 2, 3 with the amino acid sequences set forth in SEQ ID NOs: 12, 13, and 14 respectively, and the VL comprises CDRs 1, 2, 3 with the amino acid sequences set forth in SEQ ID NOs: 15, 16, and 17, respectively according to Kabat numbering.

4. The antibody or antigen-binding fragment thereof of claim 1, wherein the VH comprises CDRs 1, 2, 3 with the amino acid sequences set forth in SEQ ID NOs: 18, 19, and 20 respectively, and the VL comprises CDRs 1, 2, 3 with the amino acid sequences set forth in SEQ ID NOs: 21, 22, and 23, respectively according to Kabat numbering.

5. The antibody or antigen-binding fragment thereof of claim 1, wherein the VH comprises CDRs 1, 2, 3 with the amino acid sequences set forth in SEQ ID NOs: 24, 25, and 26, respectively, and the VL comprises CDRs 1, 2, 3 with the amino acid sequences set forth in SEQ ID NOs: 27, 28, and 29, respectively according to Kabat numbering.

6. The antibody or antigen-binding fragment thereof of claim 1, wherein the VH comprises CDRs 1, 2, 3 with the amino acid sequences set forth in SEQ ID NOs: 30, 31, and 32, respectively, and the VL comprises CDRs 1, 2, 3 with the amino acid sequences set forth in SEQ ID NOs: 33, 34, and 35, respectively according to Kabat numbering.

7. The antibody or antigen-binding fragment thereof of any one of claims 1-6, wherein the antibody or antigen-binding fragment specifically binds to human TNFR2.

8. The antibody or antigen-binding fragment thereof of any one of claims 1-7, wherein the antibody or antigen-binding fragment is a human antibody or antigen-binding fragment thereof (e.g., a human IgG1 antibody).

9. The antibody or antigen-binding fragment thereof of any one of claims 1-8, wherein the antibody or antigen-binding fragment is a single-chain variable fragment (scFV).

10. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 7, and 8, respectively, and wherein the VH, when paired with a light chain variable region (VL) comprising the amino acid sequence set forth in SEQ ID NO: 37 binds to TNFR2;
(2) an immunoglobulin light chain or a fragment thereof comprising a VL comprising CDRs 1, 2, and 3 comprising the amino acid sequences set forth in SEQ ID NOs: 9, 10, and 11, respectively, and wherein the VL, when paired with a VH comprising the amino acid sequence set forth in SEQ ID NO: 36 binds to TNFR2;
(3) an immunoglobulin heavy chain or a fragment thereof comprising a heavy chain variable region (VH) comprising CDRs 1, 2, and 3 comprising the amino acid sequences set forth in SEQ ID NOs: 12, 13, and 14, respectively, and wherein the VH, when paired with a light chain variable region (VL) comprising the amino acid sequence set forth in SEQ ID NO: 39 binds to TNFR2;
(4) an immunoglobulin light chain or a fragment thereof comprising a VL comprising CDRs 1, 2, and 3 comprising the amino acid sequences set forth in SEQ ID NOs: 15, 16, and 17, respectively, and wherein the VL, when paired with a VH comprising the amino acid sequence set forth in SEQ ID NO: 38 binds to TNFR2;
(5) an immunoglobulin heavy chain or a fragment thereof comprising a heavy chain variable region (VH) comprising CDRs 1, 2, and 3 comprising the amino acid sequences set forth in SEQ ID NOs: 18, 19, and 20, respectively, and wherein the VH, when paired with a light chain variable region (VL) comprising the amino acid sequence set forth in SEQ ID NO: 41 binds to TNFR2;
(6) an immunoglobulin light chain or a fragment thereof comprising a VL comprising CDRs 1, 2, and 3 comprising the amino acid sequences set forth in SEQ ID NOs: 21, 22, and 23, respectively, and wherein the VL, when paired with a VH comprising the amino acid sequence set forth in SEQ ID NO: 40 binds to TNFR2;
(7) an immunoglobulin heavy chain or a fragment thereof comprising a heavy chain variable region (VH) comprising CDRs 1, 2, and 3 comprising the amino acid sequences set forth in SEQ ID NOs: 24, 25, and 26, respectively, and wherein the VH, when paired with a light chain variable region (VL) comprising the amino acid sequence set forth in SEQ ID NO: 73 binds to TNFR2;
(8) an immunoglobulin light chain or a fragment thereof comprising a VL comprising CDRs 1, 2, and 3 comprising the amino acid sequences set forth in SEQ ID NOs: 27, 28, and 29, respectively, and wherein the VL, when paired with a VH comprising the amino acid sequence set forth in SEQ ID NO: 72 binds to TNFR2;
(9) an immunoglobulin heavy chain or a fragment thereof comprising a heavy chain variable region (VH) comprising CDRs 1, 2, and 3 comprising the amino acid sequences set forth in SEQ ID NOs: 30, 31, and 32, respectively, and wherein the VH, when paired with a light chain variable region (VL) comprising the amino acid sequence set forth in SEQ ID NO: 75 binds to TNFR2; or
(10) an immunoglobulin light chain or a fragment thereof comprising a VL comprising CDRs 1, 2, and 3 comprising the amino acid sequences set forth in SEQ ID NOs: 33, 34, and 35, respectively, and wherein the VL, when paired with a VH comprising the amino acid sequence set forth in SEQ ID NO: 74 binds to TNFR2.

11. The nucleic acid of claim 10, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 6, 7, and 8, respectively.

12. The nucleic acid of claim 10, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 9, 10, and 11, respectively.

13. The nucleic acid of claim 10, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 12, 13, and 14, respectively.

14. The nucleic acid of claim 10, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 15, 16, and 17, respectively.

15. The nucleic acid of claim 10, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 18, 19, and 20, respectively.

16. The nucleic acid of claim 10, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 21, 22, and 23, respectively.

17. The nucleic acid of claim 10, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 24, 25, and 26, respectively.

18. The nucleic acid of claim 10, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 27, 28, and 29, respectively.

19. The nucleic acid of claim 10, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 30, 31, and 32, respectively.

20. The nucleic acid of claim 10, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 33, 34, and 35, respectively.

21. The nucleic acid of any one of claims 10-20, wherein the VH when paired with a VL specifically binds to human TNFR2, or the VL when paired with a VH specifically binds to human TNFR2.

22. The nucleic acid of any one of claims 10-21, wherein the immunoglobulin heavy chain or the fragment thereof is a human immunoglobulin heavy chain or a fragment thereof, and the immunoglobulin light chain or the fragment thereof is a human immunoglobulin light chain or a fragment thereof.

23. The nucleic acid of any one of claims 10-22, wherein the nucleic acid encodes a single-chain variable fragment (scFv).

24. The nucleic acid of any one of claims 10-23, wherein the nucleic acid is cDNA.

25. A vector comprising one or more of the nucleic acids of any one of claims 10-24.

26. A vector comprising two of the nucleic acids of any one of claims 10-24, wherein the vector encodes the VH region and the VL region that together bind to TNFR2.

27. A pair of vectors, wherein each vector comprises one of the nucleic acids of any one of claims 10-24, wherein together the pair of vectors encodes the VH region and the VL region that together bind to TNFR2.

28. A cell comprising the vector of claim 25 or 26, or the pair of vectors of claim 27.

29. The cell of claim 28, wherein the cell is a CHO cell.

30. A cell comprising one or more of the nucleic acids of any one of claims 10-24.

31. A cell comprising two of the nucleic acids of any one of claims 10-24.

32. The cell of claim 31, wherein the two nucleic acids together encode the VH region and the VL region that together bind to TNFR2.

33. A method of producing an antibody or an antigen-binding fragment thereof, the method comprising
(a) culturing the cell of any one of claims 28-32 under conditions sufficient for the cell to produce the antibody or the antigen-binding fragment; and
(b) collecting the antibody or the antigen-binding fragment produced by the cell.

34. An antibody or antigen-binding fragment thereof that binds to TNFR2 comprising a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 36, and the selected VL sequence is SEQ ID NO: 37;
(2) the selected VH sequence is SEQ ID NO: 38, and the selected VL sequence is SEQ ID NO: 39;
(3) the selected VH sequence is SEQ ID NO: 40, and the selected VL sequence is SEQ ID NO: 41;
(4) the selected VH sequence is SEQ ID NO: 72, and the selected VL sequence is SEQ ID NO: 73; or
(5) the selected VH sequence is SEQ ID NO: 74, and the selected VL sequence is SEQ ID NO: 75.

35. The antibody or antigen-binding fragment thereof of claim 34, wherein the VH comprises the sequence of SEQ ID NO: 36 and the VL comprises the sequence of SEQ ID NO: 37.

36. The antibody or antigen-binding fragment thereof of claim 34, wherein the VH comprises the sequence of SEQ ID NO: 72 and the VL comprises the sequence of SEQ ID NO: 73.

37. The antibody or antigen-binding fragment thereof of claim 34, wherein the VH comprises the sequence of SEQ ID NO: 38 and the VL comprises the sequence of SEQ ID NO: 39.

38. The antibody or antigen-binding fragment thereof of claim 34, wherein the VH comprises the sequence of SEQ ID NO: 40 and the VL comprises the sequence of SEQ ID NO: 41.

39. The antibody or antigen-binding fragment thereof of claim 34, wherein the VH comprises the sequence of SEQ ID NO: 74 and the VL comprises the sequence of SEQ ID NO: 75.

40. The antibody or antigen-binding fragment thereof of any one of claims 34-39, wherein the antibody or antigen-binding fragment specifically binds to human TNFR2.

41. The antibody or antigen-binding fragment thereof of any one of claims 34-40, wherein the antibody or antigen-binding fragment is a human antibody or antigen-binding fragment thereof.

42. The antibody or antigen-binding fragment thereof of any one of claims 34-41, wherein the antibody or antigen-binding fragment is a single-chain variable fragment (scFV).

43. An antibody or antigen-binding fragment thereof that cross-competes with the antibody or antigen-binding fragment thereof of any one of claims 1-9 and 34-42.

44. An antibody or antigen-binding fragment thereof that binds to TNFR2 comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 36, and the selected VL sequence is SEQ ID NO: 37;
(2) the selected VH sequence is SEQ ID NO: 38, and the selected VL sequence is SEQ ID NO: 39;
(3) the selected VH sequence is SEQ ID NO: 40, and the selected VL sequence is SEQ ID NO: 41;
(4) the selected VH sequence is SEQ ID NO: 72, and the selected VL sequence is SEQ ID NO: 73; or
(5) the selected VH sequence is SEQ ID NO: 74, and the selected VL sequence is SEQ ID NO: 75.

45. An antibody-drug conjugate comprising the antibody or antigen-binding fragment thereof of any one of claims 1-9 and 34-44 covalently bound to a therapeutic agent.

46. The antibody drug conjugate of claim 45, wherein the therapeutic agent is a cytotoxic or cytostatic agent.

47. A method of treating a subject having cancer, the method comprising administering a therapeutically effective amount of a composition comprising the antibody or antigen-binding fragment thereof of any one of claims 1-9 and 34-44, or the antibody-drug conjugate of claim 45 or 46, to the subject.

48. The method of claim 47, wherein the subject has colorectal cancer, ovarian cancer, acute myeloid leukemia, Lewis lung carcinoma, breast carcinoma, hepatocellular carcinoma, neurologic cancer, glioma and colon cancer.

49. The method of claim 47, wherein the subject has renal cell carcinoma, multiple myeloma, colon cancer, ovarian cancer, glioma or cutaneous T-cell lymphomas.

50. The method of claim 47, wherein the cancer is colon cancer, glioma or ovarian cancer.

51. A method of decreasing the rate of tumor growth, the method comprising contacting a tumor cell with an effective amount of a composition comprising an antibody or antigen-binding fragment thereof of any one of claims 1-9 and 34-44, or the antibody-drug conjugate of claim 45 or 46.

52. A method of killing a tumor cell, the method comprising contacting a tumor cell with an effective amount of a composition comprising the antibody or antigen-binding fragment thereof of any one of claims 1-9 and 34-44, or the antibody-drug conjugate of claim 45 or 46.

53. A pharmaceutical composition comprising the antibody or antigen-binding fragment thereof of any one of claims 1-9 and 34-44, and a pharmaceutically acceptable carrier.

54. A pharmaceutical composition comprising the antibody drug conjugate of claim 45 or 46, and a pharmaceutically acceptable carrier.


SEQUENCE LISTING (90 sequences)

SEQ ID NO: 1 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 2 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 3 - protein, 463 aa, Macaca fascicularis
GHHSASWGAMGVQLVETWASGMAEPRAVAFTPYAPEPGGTCRLREYYDQTAQMCCSKCPPGQHAKVFCTKTSDTVCDSCEDSTYTQLWNWVPECLSCGSRCSSDQVETQACTREQNRICTCRPGWYCALSKQEGCRLCAQLRKCRPGFGVARPGTETSDVVCKPCAPGTFSNTTSSTDICRPHQICHVVAIPGNASMDAVCTSTSPTRSMAPGAVHLPQPVSTRSQHTQPTPAPSTAPGTSFLLPVGPSPPAEGSTGDIVLPVGLIVGVTALGLLIIGVVNCVIMTQVKKKPLCLQRETKVPHLPADKARGAQGPEQQHLLTTVPSSSSSSLESSASALDRRAPTRNQPQAPGAEKASGAGEARASTGSSDSSPGGHGTQVNVTCIVNVCSSSDHSSQCSSQASSTMGDTDASPSGSPKDEQVPFSKEESAFRSQLETPETLLGSTEEKPLPLGVPDAGMKPS

SEQ ID NO: 4 - protein, 482 aa, Canis lupus
MTYVGAGVNEGTHEVPSAVCIQQVFILGPRPCPHLSDSVNRATQLPYVPDPELGSSCQQSEYFDQRTQMCCSMCPPGSHARLFCTKTSNTVCARCENSTYTQLWNWVPECLSCGSRCGADQVETQACTREQNRICSCKSGWYCTLRRQGGCRLCAPLRRCRPGFGVAKPGTATSDVVCAPCAPGTFSNTTSSTDTCRPHRICSSVAVPGNASVDAVCSPAPPTVRTAPRPASTRQPGSTQPRPAEPTPGPSTPPRTSVLFPAVPSPPAEGLSTGDISLPIGLIVGVTTLGLLLIGLVNCVIVTQKKKKPFCLQGEAKVPHLPADKAHGGPGPEQQHLLTTAPSSSSSSLESAASSADGRAPPRAQPPAPGTGKAHGSGEAQASSSSSEPSCGGHGTQVNVTCIVNVCSSSGSDHGPQCSSQASHTTGDVDAGPSSSPDDQQVPFSQEECPFQFQPGALETLLENPEDKPLPLGVPDAGMKSS

SEQ ID NO: 5 - protein, 473 aa, synthetic construct
MAPAALWVALVFELQLWATGHTVPAQVVLTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 6 - protein, 7 aa, synthetic construct
SNSAAWN

SEQ ID NO: 7 - protein, 18 aa, synthetic construct
RTYYRSKWYNDYALSVKS

SEQ ID NO: 8 - protein, 13 aa, synthetic construct
EDGVGGTKDYFDY

SEQ ID NO: 9 - protein, 11 aa, synthetic construct
QASQDIDNYLN

SEQ ID NO: 10 - protein, 7 aa, synthetic construct
DVSNLEI

SEQ ID NO: 11 - protein, 9 aa, synthetic construct
QQYDNLPLT

SEQ ID NO: 12 - protein, 5 aa, synthetic construct
NFGMH

SEQ ID NO: 13 - protein, 17 aa, synthetic construct
VIWYEGSDKYYADSVKG

SEQ ID NO: 14 - protein, 13 aa, synthetic construct
DSSWGFLLYGMDV

SEQ ID NO: 15 - protein, 16 aa, synthetic construct
RSSRSLEYSDGNTYLN

SEQ ID NO: 16 - protein, 7 aa, synthetic construct
KVSNRDS

SEQ ID NO: 17 - protein, 9 aa, synthetic construct
MQGTHWPPT

SEQ ID NO: 18 - protein, 5 aa, synthetic construct
IYGMH

SEQ ID NO: 19 - protein, 17 aa, synthetic construct
LISYDGNDKYFADSVKG

SEQ ID NO: 20 - protein, 12 aa, synthetic construct
AVDTAVVTFFDY

SEQ ID NO: 21 - protein, 11 aa, synthetic construct
RASQSVSSNLA

SEQ ID NO: 22 - protein, 7 aa, synthetic construct
SASTRAT

SEQ ID NO: 23 - protein, 9 aa, synthetic construct
QQYSNWPFT

SEQ ID NO: 24 - protein, 7 aa, synthetic construct
SDSSAWN

SEQ ID NO: 25 - protein, 18 aa, synthetic construct
RTYYRSKWYNDYTVSVKS

SEQ ID NO: 26 - protein, 13 aa, synthetic construct
EDGVGGTKDYFDY

SEQ ID NO: 27 - protein, 11 aa, synthetic construct
QASQDISNYLN

SEQ ID NO: 28 - protein, 7 aa, synthetic construct
DASNLET

SEQ ID NO: 29 - protein, 9 aa, synthetic construct
QQYDNLPIT

SEQ ID NO: 30 - protein, 5 aa, synthetic construct
GDYWS

SEQ ID NO: 31 - protein, 16 aa, synthetic construct
EINHSGSTNYNPSLKS

SEQ ID NO: 32 - protein, 13 aa, synthetic construct
GHWNYDYYYGMDV

SEQ ID NO: 33 - protein, 16 aa, synthetic construct
RSSQSLLHSNGYNYLD

SEQ ID NO: 34 - protein, 7 aa, synthetic construct
LNSNRAS

SEQ ID NO: 35 - protein, 9 aa, synthetic construct
MQARQTPLT

SEQ ID NO: 36 - protein, 125 aa, synthetic construct
QVQLQQSGPGLVKPSQTLSLTCAISGDSVSSNSAAWNWIRQSPSRGLEWLGRTYYRSKWYNDYALSVKSRITINPETSKNQFSLQLNSVTPEDTAVYYCAREDGVGGTKDYFDYWGQGTLVTVSS

SEQ ID NO: 37 - protein, 107 aa, synthetic construct
DIQMTQSPSSLSASVGDRVTITCQASQDIDNYLNWYQQKPGKAPKLLIYDVSNLEIGVPSRFSGSGSGTDFTFTISSLQPEDIATYYCQQYDNLPLTFGQGTRLEIK

SEQ ID NO: 38 - protein, 122 aa, synthetic construct
QVQLVESGGGVVQPGRSLRLSCAASGFTFSNFGMHWVRQAPGKGLEWVAVIWYEGSDKYYADSVKGRFTISRDNSKNTLYLQMNSLRAEDTAVYYCARDSSWGFLLYGMDVWGQGTTVTVSS

SEQ ID NO: 39 - protein, 112 aa, synthetic construct
DVVMTQSPLSLPVTLGQPASISCRSSRSLEYSDGNTYLNWFQQRPGQSPRRLIYKVSNRDSGVPDRFSGSGSGTAFTLKISGVEAEDVGVYYCMQGTHWPPTFGGGTKVEIK

SEQ ID NO: 40 - protein, 121 aa, synthetic construct
QVQLVESGGGVVHPGKSLRLSCAASGFTFNIYGMHWVRQAPGKGLEWVALISYDGNDKYFADSVKGRFTISRDNSKNTLYLQMNSLRAEDTAVYYCAKAVDTAVVTFFDYWGQGTLVTVSS

SEQ ID NO: 41 - protein, 107 aa, synthetic construct
EIVMTQSPVTLSVSPGERATLSCRASQSVSSNLAWYQQKPGQAPRLLIYSASTRATGIPARFSGSGSGTEFTLTISSLQSEDLAVYYCQQYSNWPFTFGPGTKVDIK

SEQ ID NO: 42 - protein, 12 aa, synthetic construct
GDSVSSNSAAWN

SEQ ID NO: 43 - protein, 7 aa, synthetic construct
YYRSKWY

SEQ ID NO: 44 - protein, 13 aa, synthetic construct
EDGVGGTKDYFDY

SEQ ID NO: 45 - protein, 11 aa, synthetic construct
QASQDIDNYLN

SEQ ID NO: 46 - protein, 7 aa, synthetic construct
DVSNLEI

SEQ ID NO: 47 - protein, 9 aa, synthetic construct
QQYDNLPLT

SEQ ID NO: 48 - protein, 10 aa, synthetic construct
GFTFSNFGMH

SEQ ID NO: 49 - protein, 6 aa, synthetic construct
WYEGSD

SEQ ID NO: 50 - protein, 13 aa, synthetic construct
DSSWGFLLYGMDV

SEQ ID NO: 51 - protein, 16 aa, synthetic construct
RSSRSLEYSDGNTYLN

SEQ ID NO: 52 - protein, 7 aa, synthetic construct
KVSNRDS

SEQ ID NO: 53 - protein, 9 aa, synthetic construct
MQGTHWPPT

SEQ ID NO: 54 - protein, 10 aa, synthetic construct
GFTFNIYGMH

SEQ ID NO: 55 - protein, 6 aa, synthetic construct
SYDGND

SEQ ID NO: 56 - protein, 12 aa, synthetic construct
AVDTAVVTFFDY

SEQ ID NO: 57 - protein, 11 aa, synthetic construct
RASQSVSSNLA

SEQ ID NO: 58 - protein, 7 aa, synthetic construct
SASTRAT

SEQ ID NO: 59 - protein, 9 aa, synthetic construct
QQYSNWPFT

SEQ ID NO: 60 - protein, 12 aa, synthetic construct
GDSVSSDSSAWN

SEQ ID NO: 61 - protein, 7 aa, synthetic construct
YYRSKWY

SEQ ID NO: 62 - protein, 13 aa, synthetic construct
EDGVGGTKDYFDY

SEQ ID NO: 63 - protein, 11 aa, synthetic construct
QASQDISNYLN

SEQ ID NO: 64 - protein, 7 aa, synthetic construct
DASNLET

SEQ ID NO: 65 - protein, 9 aa, synthetic construct
QQYDNLPIT

SEQ ID NO: 66 - protein, 10 aa, synthetic construct
DGSFSGDYWS

SEQ ID NO: 67 - protein, 5 aa, synthetic construct
NHSGS

SEQ ID NO: 68 - protein, 13 aa, synthetic construct
GHWNYDYYYGMDV

SEQ ID NO: 69 - protein, 16 aa, synthetic construct
RSSQSLLHSNGYNYLD

SEQ ID NO: 70 - protein, 7 aa, synthetic construct
LNSNRAS

SEQ ID NO: 71 - protein, 9 aa, synthetic construct
MQARQTPLT

SEQ ID NO: 72 - protein, 125 aa, synthetic construct
QVQLQQSGPGLVKPSQTLSLTCAISGDSVSSDSSAWNWIRQSPSRGLEWLGRTYYRSKWYNDYTVSVKSRITINPDTSKNQFSLQLNSVTPEDTAVYYCAREDGVGGTKDYFDYWGQGTLVTVSS

SEQ ID NO: 73 - protein, 107 aa, synthetic construct
DIQMTQSPSSLSASVGDRVTITCQASQDISNYLNWYQQKPGKAPKLLIYDASNLETGVPSRFSGSGSGTDFTFTINSLQPEDFATYFCQQYDNLPITFGQGTRLEIK

SEQ ID NO: 74 - protein, 121 aa, synthetic construct
QVQLQQWGAGLLKPSETLSLICAVYDGSFSGDYWSWIRQPPGKGLEWIGEINHSGSTNYNPSLKSRVTISVDTSKNQFSLKLSSVTAADTAVYYCARGHWNYDYYYGMDVWGQGTTVTVSS

SEQ ID NO: 75 - protein, 112 aa, synthetic construct
DIVMTQSPLSLPVTPGEPASISCRSSQSLLHSNGYNYLDWYLQKPGQSPQLLIYLNSNRASGVPDRFSGSGSGTDFTLKISRVEAEDVGLYYCMQARQTPLTFGGGTKVEIK

SEQ ID NO: 76 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 80 - protein, 118 aa, synthetic construct [sequence omitted]

SEQ ID NO: 81 - protein, 107 aa, synthetic construct [sequence omitted]

SEQ ID NO: 82 - protein, 121 aa, synthetic construct
EVQLLESGGGLVQPGGSLRLSCAASGFTFDDYGMSWVRQAPGKGLEWVSVIYSGGSTYYADSVKGRFTISRDNSKNTLYLQMNSLRAEDTAVYYCARDRSSSWYRDGMDVWGQGTLVTVSS

SEQ ID NO: 83 - protein, 112 aa, synthetic construct
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLSGWVFGGGTKLTVLG

SEQ ID NO: 84 - protein, 123 aa, synthetic construct
EVQLVESGGGLVQPGGSLRLSCAASGFSLSDYFMTWVRQAPGKGLEWVGIINTGGDSYYATWAKGRFTISRDNSKNTLYLQMNSLRAEDTAVYYCARDTGYGGYDYAGSFDPWGQGTLVTVSS

SEQ ID NO: 85 - protein, 110 aa, synthetic construct
DIQMTQSPSSVSASVGDRVTITCQASENINSWLAWYQQKPGKAPKLLIYEASKLASGVPSRFSGSGSGTDFTLTISSLQPEDFATYYCQQGYIYIDVGNIFGGGTKVEIK

SEQ ID NO: 86 - protein, 118 aa, synthetic construct
QVQLVQSGAELKKPGASVKVSCKASSYSFTDYNMNWVRQAPGQSLEWMGIIFPKYGTTSYAQKLQGRVTLTTDTSTSTAYMELRSLRSDDTAVYYCATDGGTWYFDVWGTGTTVTVSS

SEQ ID NO: 87 - protein, 106 aa, synthetic construct
DIQLTQSPSFLSASVGDRVTITCRASSSVTYMYWYQQKPGKAPKPWIYLTSNLASGVPSRFSGSGSGTEYTLTISSLQPEDAATYYCQQWSSNPPTFGSGTKLEIK

SEQ ID NO: 88 - protein, 120 aa, synthetic construct
EVQLVESGGGLVQPGGSLRLSCAASGIDLSSYAMGWVRQAPGKGLEWVGDISTSGNAYYATWVKGRFTISRDNSKNTLYLQMNSLRAEDTAVYYCARADYGGETYAFDPWGQGTLVTVSS

SEQ ID NO: 89 - protein, 110 aa, synthetic construct
DIQMTQSPSSLSASVGDRVTITCQASQSISSYLNWYQQKPGKAPKLLIYSASTLASGVPSRFSGSGSGTDFTLTISSLQPEDFATYYCQQGYSDSNIDNVFGGGTKVEIK

SEQ ID NO: 90 - protein, 330 aa, synthetic construct [sequence omitted]